(19) United States
(12) Patent Application Publication
(10) Pub. No.: US 2009/0156598 A1
(43) Pub. Date: Jun. 18, 2009

(54) Imidazolopyrimidine modulators of TRPV1

(76) Inventor: Alec D. Lebsack, San Diego, CA (US)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 12/316,827

(22) Filed: Dec. 16, 2008

Related U.S. Application Data
(60) Provisional application No. 61/014,167, filed on Dec. 17, 2007.

(51) Int. Cl. A61K 31/52; C07D 473/34; C07D 413/14; A61K 31/5377; A61K 31/496; C07D 403/14; C07D 401/14; A61P 1/00; A61P 29/00; A61P 7/00; A61P 43/00

(52) U.S. Cl. 514/234.2; 544/277; 514/263.4; 514/263.22; 544/118; 514/252.16

(57) ABSTRACT

Certain TRPV1-modulating imidazolopyrimidine compounds are described. The compounds may be used in pharmaceutical compositions and methods for treating disease states, disorders, and conditions mediated by TRPV1 activity, such as pain, arthritis, itch, cough, asthma, or inflammatory bowel disease.

This application claims the benefit of U.S. provisional patent application Ser. No. 61/014,167, filed Dec. 17, 2007, which is hereby incorporated by reference.

### FIELD OF THE INVENTION

The present invention relates to certain imidazolopyrimidine compounds, pharmaceutical compositions containing them, and methods of using them for the treatment of disease states, disorders, and conditions mediated by TRPV1 activity.

### BACKGROUND OF THE INVENTION

Transient receptor potential (TRP) channel proteins constitute a large and diverse family of proteins that are expressed in many tissues and cell types. One TRP channel protein of particular interest is the vanilloid receptor 1 (TRPV1 or VR1), a non-selective Ca⁺² channel that is the molecular target of vanilloid compounds (e.g., capsaicin and resiniferatoxin). Such vanilloid compounds are known to selectively depolarize nociceptors, specialized primary afferent neurons involved in the signaling pathway that leads to the sensation of pain. TRPV1 is activated by a diverse range of stimuli, including vanilloids, membrane depolarization, heat, stretch, low pH, inflammatory mediators (e.g., lipoxygenase metabolites), and endocannabinoid compounds. Because heightened activity of nociceptors contributes to unwanted pain, inflammatory conditions, thermoregulation, and control of smooth muscle tone and reflexes in mammals, modulation of signaling in this pathway is important in treatment and prophylaxis of various clinical syndromes (Caterina, M. J., Pain 2003, 105(1-2), 5-9; Caterina, M. J. et. al., Annu. Rev. Neurosci. 2001, 24, 487-517; Tominaga, M. et. al., J. Neurobiol. 2004, 61, 3-12; Voets, T. et. al., Nature 2004, 430, 748-754).

Because of TRPV1's connection with the sensory nervous system, TRPV1 agonists and antagonists may be therapeutically useful in the treatment or prophylaxis of disease states, disorders, and conditions mediated by TRPV1 activity, such as: i) pain (e.g., acute, chronic, inflammatory, or neuropathic pain); ii) itch (Kim et al., Neurosci. Lett. 2004, 361, 159) and various inflammatory disorders (Stucky, C. L. et. al., Neuroscience 1998, 84, 1257; Moore, B. A. et. al., Am. J. Physiol. Gastrointest. Liver Physiol. 2002, 282, G1045; Kwak, J. Y. et. al., Neuroscience 1998, 86, 619; Morris, V. H. et. al., Pain 1997, 71, 179; Greiff, L. et. al., Thorax 1995, 50, 225); iii) inner ear disorders (Balaban, C. D. et al., Hear. Res. 2003, 175, 165-70; Zheng, J. et al., J. Neurophys. 2003, 90, 444-55); iv) fever and other disorders or symptoms affected by thermoregulation (Jancso-Gabor et al., J. Physiol. 1970, 206, 495; Swanson et al., J. Med. Chem. 48, 1857; lida et al., Neurosci. Lett. 2005, 378, 28); v) tracheobronchial and diaphragmatic dysfunction; and vi) gastrointestinal and urinary tract disorders (Lazzeri, M. et al., Eur. Urology 200, 792-798; Apostolidis, A. et. al., Urology 2005, 65, 400-405). Additionally, TRPV1 modulators may be therapeutically useful in the treatment or prophylaxis of anxiety (Marsch, R. et al., J. Neurosci. 2007, 27(4), 832-839); eye-related disorders (such as glaucoma, vision loss, and increased intraocular pressure) (Calkins, D. J. et al., Abstract from ARVO 2006 Annual Meeting, Program #1557, Poster #B93); baldness (e.g., by stimulating hair growth) (Bodo, E. et al., Am. J. Pathol. 2005, 166(4), 985-998); diabetes (including insulin-resistant diabetes or diabetic conditions mediated by insulin sensitivity or secretion) (Razavi, R. et al., Cell 2006, 127(6), 1097-1099; Akiba, Y. et al., Biochem. Biophy. Res. Commun. 2004, 321(1), 219-225).

Acidosis is a well-established feature of cerebral ischaemia. Tissue pH may fall to 6 or lower, sufficient to activate TRPV1 channels expressed in the CNS. TRPV1 antagonists therefore may be useful in the treatment of disorders associated with reduced blood flow to the CNS or CNS hypoxia, such as head trauma, spinal injury, thromboembolic or hemorrhagic stroke, transient ischaemic attacks, cerebral vasospasm, hypoglycaemia, cardiac arrest, status epilepticus, perinatal asphyxia, Alzheimer's disease, and Huntington's Disease.

Certain thiazolopyrimidines have been described as CCR2b receptor antagonists (PCT Intl. Pat. Appl. Publ. WO 2005/117890), inhibitors of ATP-protein kinase interactions (U.S. Pat. Appl. Publ. 2007/0185139 (Attorney Docket No. PRD2510)), chemokine receptor antagonists (U.S. Pat. Appl. Publ. 2007/0142386; Baxter et al. Bioorg. Med. Chem. Lett. 2006, 26, 960-963), and TRPV1 modulators (U.S. patent application Ser. No. 11/824,202, filed Jun. 8, 2007). Certain thiazolopyrimidine derivatives are disclosed as growth factor receptor tyrosine kinase inhibitors in Eur. Pat. Appl. EP 1731523 (Dec. 13, 2006). Condensed heterocyclic compounds are shown as macrophage migration inhibitory factor inhibitors in JP 2001097979. Certain fused pyrimidines are described as modulators of metabotropic receptors—subtype 2 in PCT Intl. Pat. Appl. Publ. WO 2006/030031. Bicyclic pyrimidinyl derivatives are disclosed as adenosine receptor binders in U.S. Pat. Appl. Publ. US 2003/139427 and U.S. Pat. Appl. Publ. US 2002/094974. Purine derivatives are described as nerve growth promoters in PCT Intl. Pat. Appl. Publ. WO 2006/130469. Various purine analogs are disclosed as heat shock protein 90 inhibitors in U.S. Pat. Appl. Publ. 2005/0049263. Purine analogs are also described as inhibitors of cyclin dependent kinases in U.S. Pat. Appl. Publ. 2003/191086.

There remains a desire for potent TRPV1 modulators with suitable pharmaceutical properties.

### SUMMARY OF THE INVENTION

Certain imidazolopyrimidine derivatives have now been found to have TRPV1-modulating activity. In particular, the invention is directed to the general and preferred embodiments defined, respectively, by the independent and dependent claims appended hereto, which are incorporated by reference herein.

Thus, in one general aspect, the invention relates to compounds of Formula (I):

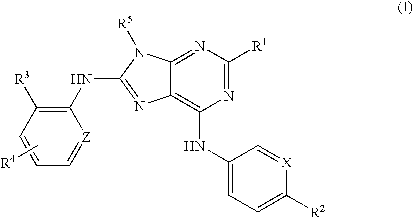

wherein:
R¹ is —H, —C₁₋₆alkyl, —OC₁₋₆alkyl, —NR^{a}R^{b}, —S—C₁₋₆alkyl, or —SO₂—C₁₋₆alkyl;
   where R^{a} and R^{b} are each independently —H, —C₁₋₆alkyl, or —CH₂-pyridinyl; or, R^{a} and R^{b} taken together with the nitrogen of attachment in —NR^{a}R^{b} form a saturated monocyclic heterocycloalkyl group unsubstituted or substituted with a —C₁₋₆alkyl substituent;
R² is —H, —C₁₋₆alkyl, —OH, —OC₁₋₆alkyl, —CN, —NO₂, —N(R^{h})Rⁱ, —C(O)N(R^{h})Rⁱ, —N(R^{h})C(O)Rⁱ, —N(R^{h})SO₂C₁₋₆alkyl, —N(SO₂C₁₋₆alkyl)₂, —C(O)C₁₋₆alkyl, —S(O)₀₋₂—C₁₋₆alkyl, —SO₂CF₃, —SO₂N(R^{h})Rⁱ, —SCF₃, halo, —CF₃, —OCF₃, —CO₂H, —CO₂C₁₋₆alkyl, —C(R^{j})₂—CN, —C(R^{j})₂—CO₂C₁₋₄alkyl, —C(Rⁱ)₂—CO₂H, —C(R^{j})₂—CON(R^{h})Rⁱ, —C(R^{j})₂—CH₂N(R^{h})Rⁱ, or —C(R^{j})₂—OH;
   where R^{h} and Rⁱ are each independently —H or —C₁₋₆alkyl; or R^{h} and Rⁱ taken together with their nitrogen of attachment in —NR^{h}Rⁱ form a saturated monocyclic heterocycloalkyl group unsubstituted or substituted with methyl;
   where each R^{j} is independently —H or —C₁₋₆alkyl;
X and Z are each independently N or CR^{m}, where R^{m} is —H, halo, or —CF₃;
R³ is —CF₃, halo, —CN, —CO₂H, —CO₂C₁₋₆alkyl, —C(O)N(R^{k})R^{l}, —C₁₋₄alkyl-OH, —C₁₋₄alkyl-N(R^{k})R^{l}, —S(O)₀₋₂—C₁₋₆alkyl, —SO₂CF₃, or —SO₂N(R^{k})R^{l};
   where R^{k} and R^{l} are each independently —H or —C₁₋₆alkyl;
R⁴ is —H, —CF₃, halo, —CN, —CO₂H, —CO₂C₁₋₆alkyl, —C(O)N(Rⁿ)R^{o}, —C₁₋₄alkyl-OH, —C₁₋₄alkyl-N(Rⁿ)R^{o}, —S(O)₀₋₂—C₁₋₆alkyl, —SO₂CF₃, or —SO₂N(Rⁿ)R^{o};
   where Rⁿ and R^{o} are each independently —H or —C₁₋₆alkyl; and
R⁵ is —H or —CH₃.

The invention also relates to pharmaceutically acceptable salts, pharmaceutically acceptable prodrugs, and pharmaceutically active metabolites of compounds of Formula (I). In certain preferred embodiments, the compound of Formula (I) is a compound selected from those species described or exemplified in the detailed description below.

In a further general aspect, the invention relates to pharmaceutical compositions each comprising: (a) an effective amount of an agent selected from compounds of Formula (I) and pharmaceutically acceptable salts, pharmaceutically acceptable prodrugs, and pharmaceutically active metabolites thereof; and (b) a pharmaceutically acceptable excipient.

In another general aspect, the invention is directed to a method of treating a subject suffering from or diagnosed with a disease, disorder, or medical condition (collectively, “indications”) mediated by TRPV1 activity, comprising administering to the subject in need of such treatment an effective amount of a compound of Formula (I), or a pharmaceutically acceptable salt, pharmaceutically acceptable prodrug, or pharmaceutically active metabolite of such compound. In certain preferred embodiments of the inventive method, the disease, disorder, or medical condition is selected from: pain (acute, chronic, inflammatory, or neuropathic pain); itch or various inflammatory disorders; inner ear disorders; fever and other conditions or disorders of thermoregulation; tracheobronchial and diaphragmatic dysfunction; gastrointestinal and urinary tract disorders; and disorders associated with reduced blood flow to the CNS or CNS hypoxia.

Preferred embodiments, features, and advantages of the invention will be apparent from the following detailed description and through practice of the invention.

### DETAILED DESCRIPTION OF INVENTION AND ITS PREFERRED EMBODIMENTS

The invention may be more fully appreciated by reference to the following detailed description, including the following glossary of terms and the concluding examples. For the sake of brevity, the disclosures of the publications, including patents, cited in this specification are herein incorporated by reference.

The terms “including”, “containing” and “comprising” are used herein in their open, non-limiting sense.

The term “alkyl” refers to a straight- or branched-chain alkyl group having from 1 to 12 carbon atoms in the chain. Examples of alkyl groups include methyl (Me, which also may be structurally depicted by a/symbol), ethyl (Et), n-propyl (Pr), isopropyl (iPr), butyl (nBu), isobutyl (iBu), sec-butyl (sBu), tert-butyl (tBu), pentyl, isopentyl, tert-pentyl, hexyl, isohexyl, and so on.

The term “cycloalkyl” refers to a saturated or partially saturated, monocyclic, fused polycyclic, or spiro polycyclic carbocycle having from 3 to 12 ring atoms per carbocycle. Illustrative examples of cycloalkyl groups include the following entities (depicted without their bonds of attachment):

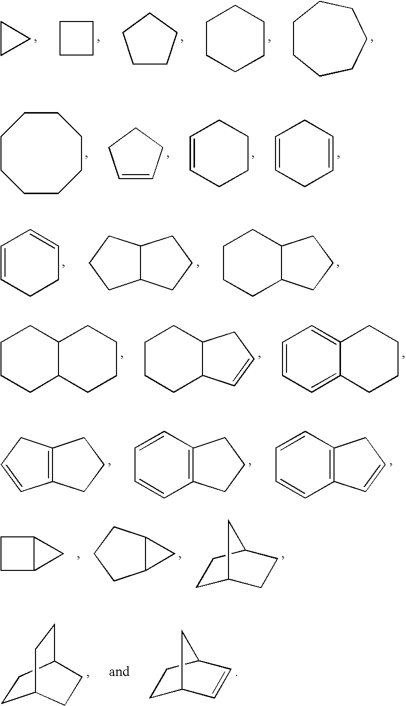

A “heterocycloalkyl” refers to a monocyclic, or fused, bridged, or spiro polycyclic ring structure that is saturated or partially saturated and has from 3 to 12 ring atoms per ring structure selected from carbon atoms and up to three heteroatoms selected from nitrogen, oxygen, and sulfur. The ring structure may optionally contain up to two oxo groups on carbon or sulfur ring members. Illustrative examples (depicted without their bonds of attachment) include:

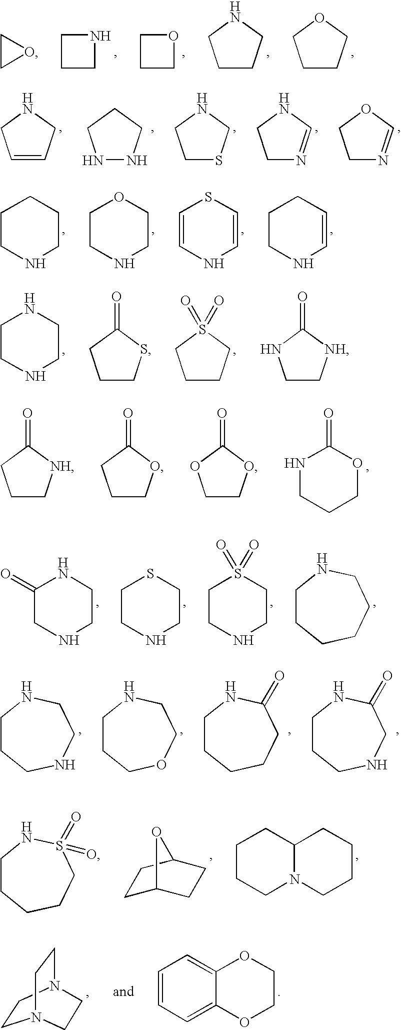

The term “heteroaryl” refers to a monocyclic, fused bicyclic, or fused polycyclic aromatic heterocycle (ring structure having ring atoms selected from carbon atoms and up to four heteroatoms selected from nitrogen, oxygen, and sulfur) having from 3 to 12 ring atoms per heterocycle. Illustrative examples of heteroaryl groups include the following entities (depicted without their bonds of attachment):

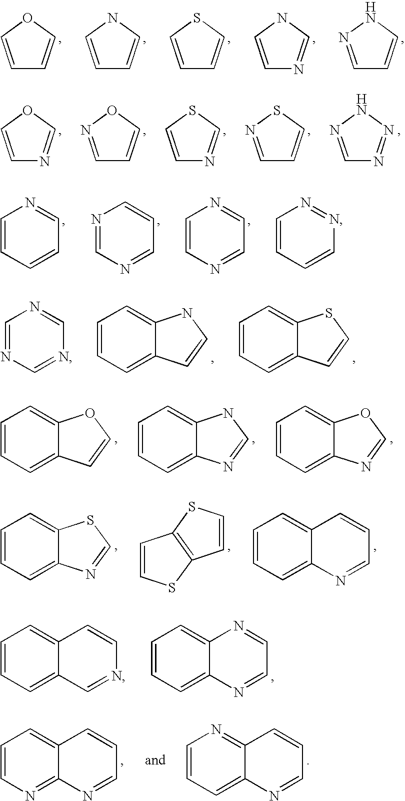

Those skilled in the art will recognize that the species of cycloalkyl, heterocycloalkyl, and heteroaryl groups listed or illustrated above are not exhaustive, and that additional species within the scope of these defined terms may also be selected.

The term “halogen” represents chlorine, fluorine, bromine or iodine. The term “halo” represents chloro, fluoro, bromo or iodo.

The term “substituted” means that the specified group or moiety bears one or more substituents. The term “unsubstituted” means that the specified group bears no substituents. The term “optionally substituted” means that the specified group is unsubstituted or substituted by one or more substituents. Where the term “substituted” is used to describe a structural system, the substitution is meant to occur at any valency-allowed position on the system. In cases where a specified moiety or group is not expressly noted as being optionally substituted or substituted with any specified substituent, it is understood that such a moiety or group is intended to be unsubstituted.

Any formula given herein is intended to represent compounds having structures depicted by the structural formula as well as certain variations or forms. In particular, compounds of any formula given herein may have asymmetric centers and therefore exist in different enantiomeric forms. All optical isomers and stereoisomers of the compounds of any general structural formula, and mixtures thereof, are considered within the scope of the formula. Thus, any general formula given herein is intended to represent a racemate, one or more enantiomeric forms, one or more diastereomeric forms, one or more atropisomeric forms, and mixtures thereof. Furthermore, certain structures may exist as geometric isomers (i.e., cis and trans isomers), as tautomers, or as atropisomers. Additionally, any general formula given herein is intended to embrace hydrates, solvates, and polymorphs of such compounds, and mixtures thereof.

Any general formula given herein is also intended to represent unlabeled forms as well as isotopically labeled forms of the compounds. Isotopically labeled compounds have structures of the formulas given herein except that one or more atoms are replaced by an atom having a selected atomic mass or mass number. Examples of isotopes that can be incorporated into compounds of the invention include isotopes of hydrogen, carbon, nitrogen, oxygen, phosphorous, fluorine, chlorine, and iodine, such as ²H, ³H, ¹¹C, ¹³C, ¹⁴C, ¹⁵N, ¹⁸O, ¹⁷O, ³¹P, ³²P, ³⁵S, ¹⁸F, ³⁶Cl, and ¹²⁵I, respectively. Such isotopically labeled compounds are useful in metabolic studies (preferably with 14C), reaction kinetic studies (with, for example 2H or 3H), detection or imaging techniques (such as positron emission tomography (PET) or single-photon emission computed tomography (SPECT)) including drug or substrate tissue distribution assays, or in radioactive treatment of patients. In particular, an ¹⁸F or ¹¹C labeled compound may be particularly preferred for PET or SPECT studies. Further, substitution with heavier isotopes such as deuterium (i.e., ²H) may afford certain therapeutic advantages resulting from greater metabolic stability, for example increased in vivo half-life or reduced dosage requirements. Isotopically labeled compounds of this invention and prodrugs thereof can generally be prepared by carrying out the procedures disclosed in the schemes or in the examples and preparations described below by substituting a readily available isotopically labeled reagent for a non-isotopically labeled reagent.

When referring to a formula given herein, the selection of a particular moiety from a list of possible species for a specified variable is not intended to define the moiety for the variable appearing elsewhere. In other words, where a variable appears more than once in a formula, the choice of the species from a specified list is independent of the choice of the species for the same variable elsewhere in the formula unless otherwise indicated.

In certain preferred embodiments of compounds of Formula (I), R¹ is —H, methyl, methanesulfanyl, methanesulfonyl, or methoxy. In other embodiments, R¹ is isopropylamino, isobutylamino, or (pyridin-2-ylmethyl)amino, or a pyrrolidin-1-yl, piperidin-1-yl, morpholin-4-yl, or piperazin-1-yl group unsubstituted or substituted with a —C₁₋₄alkyl substituent.

In preferred embodiments, R² is —H, methyl, isopropyl, tert-butyl, —OCH₃, —SO₂CH₃, —SO₂CF₃, —SO₂NH₂, —SO₂(morpholinyl), —SO₂(piperazinyl), fluoro, chloro, —CF₃, —OCF₃, —CO₂CH₃, —C(CH₃)₂—CN, —C(CH₃)₂—CO₂CH₃, —C(CH₃)₂—CONH₂, or —C(CH₃)₂—OH. In other preferred embodiments, R² is —H, —CF₃, tert-butyl, or methanesulfonyl. In still other preferred embodiments, R² is —CF₃.

In preferred embodiments, X is CR^{m}, where R^{m} is —H, chloro, or fluoro. In other embodiments, X is CR^{m}, where R^{m} is —H. In other embodiments, X is N.

In preferred embodiments, Z is CR^{m}, where R^{m} is —H, chloro, or —CF₃. In other preferred embodiments, Z is N.

In preferred embodiments, R³ is —CF₃, halo, —CN, —C(O)N(R^{k})R^{l}, —CH₂OH, or —CH₂N(R^{k})R^{l}. In preferred embodiments, R³ is —CF₃ or halo.

In preferred embodiments, R⁴ is —H, —CN, —C(O)N(Rⁿ)R^{o}, —CH₂OH, or —CH₂N(Rⁿ)R^{o}. In other embodiments, R⁴ is —H.

In preferred embodiments, R⁵ is —H.

In preferred embodiments, R^{a} and R^{b} are each independently —H, methyl, ethyl, isopropyl, isobutyl, or pyridinylmethyl. In other preferred embodiments, R^{a} and R^{b} are each independently —H, methyl, ethyl, isopropyl, or isobutyl. In other preferred embodiments, R^{a} and R^{b} taken together with the nitrogen of attachment form an azetidinyl, pyrrolidinyl, piperidinyl, 2-oxo-piperidin-1-yl, piperazinyl, oxo-piperazinyl, morpholinyl, thiomorpholinyl, 1,1-dioxo-1λ⁶-thiomorpholin-4-yl, or azepanyl group unsubstituted or substituted with a —C₁₋₄alkyl substituent. In further preferred embodiments, R^{a} and R^{b} taken together with the nitrogen of attachment form an azetidinyl, pyrrolidinyl, piperidinyl, piperazinyl, or morpholinyl group, each unsubstituted or substituted with a methyl, isopropyl, or isobutyl substituent.

Preferably, R^{h} and Rⁱ are each independently —H or methyl; or R^{h} and Rⁱ taken together with their nitrogen of attachment form a morpholinyl or piperazinyl group, unsubstituted or substituted with methyl.

In some preferred embodiments, R^{j} is —H or methyl.

In preferred embodiments, R^{k} and R^{l} are each independently —H or methyl.

Further preferred embodiments of Formula (I) include compounds wherein combinations of two or more of the preferred embodiments for each of R¹⁻⁵, X, Z, R^{a-b}, and R^{h-o}, listed above are selected.

The invention includes also pharmaceutically acceptable salts of the compounds represented by Formula (I), preferably of those described above. Pharmaceutically acceptable salts of the specific compounds exemplified herein are especially preferred.

A “pharmaceutically acceptable salt” is intended to mean a salt of a free acid or base of a compound represented by Formula (I) that is pharmacologically effective and suitable for administration to the subject such that contact with the tissues of patients occurs without undue toxicity, irritation, or allergic response. See generally, Berge et al., “Pharmaceutical Salts”, J. Pharm. Sci., 1977, 66:1-19, and Handbook of Pharmaceutical Salts, Properties, Selection, and Use, Stahl and Wermuth, Eds., Wiley-VCH and VHCA, Zurich, 2002.

A compound may possess a sufficiently acidic group, a sufficiently basic group, or both types of functional groups, and accordingly react with an inorganic or organic bases, or an inorganic and organic acid, to form a pharmaceutically acceptable salt. Examples of pharmaceutically acceptable salts include sulfates, pyrosulfates, bisulfates, sulfites, bisulfites, phosphates, monohydrogen-phosphates, dihydrogenphosphates, metaphosphates, pyrophosphates, chlorides, bromides, iodides, acetates, propionates, decanoates, caprylates, acrylates, formates, isobutyrates, caproates, heptanoates, propiolates, oxalates, malonates, succinates, suberates, sebacates, fumarates, maleates, butyne-1,4-dioates, hexyne-1,6-dioates, benzoates, chlorobenzoates, methylbenzoates, dinitrobenzoates, hydroxybenzoates, methoxybenzoates, phthalates, sulfonates, xylenesulfonates, phenylacetates, phenylpropionates, phenylbutyrates, citrates, lactates, γ-hydroxybutyrates, glycolates, tartrates, methane-sulfonates, propanesulfonates, naphthalene-1-sulfonates, naphthalene-2-sulfonates, and mandelates.

If the compound contains a basic nitrogen, the desired pharmaceutically acceptable salt may be prepared by any suitable method available in the art, for example, treatment of the free base with an inorganic acid, such as hydrochloric acid, hydrobromic acid, sulfuric acid, sulfamic acid, nitric acid, boric acid, phosphoric acid, and the like, or with an organic acid, such as acetic acid, phenylacetic acid, propionic acid, stearic acid, lactic acid, ascorbic acid, maleic acid, hydroxymaleic acid, isethionic acid, succinic acid, valeric acid, fumaric acid, malonic acid, pyruvic acid, oxalic acid, glycolic acid, salicylic acid, oleic acid, palmitic acid, lauric acid, a pyranosidyl acid, such as glucuronic acid or galacturonic acid, an alpha-hydroxy acid, such as mandelic acid, citric acid, or tartaric acid, an amino acid, such as aspartic acid or glutamic acid, an aromatic acid, such as benzoic acid, 2-acetoxybenzoic acid, naphthoic acid, or cinnamic acid, a sulfonic acid, such as laurylsulfonic acid, p-toluenesulfonic acid, methanesulfonic acid, ethanesulfonic acid, and any compatible mixture of acids such as those given as examples herein.

If the compound is an acid, such as a carboxylic acid or sulfonic acid, the desired pharmaceutically acceptable salt may be prepared by any suitable method, for example, treatment of the free acid with an inorganic or organic base, such as an amine (primary, secondary or tertiary), an alkali metal hydroxide, alkaline earth metal hydroxide, any compatible mixture of bases such as those given as examples herein. Illustrative examples of suitable salts include organic salts derived from amino acids, such as glycine and arginine, ammonia, carbonates, bicarbonates, primary, secondary, and tertiary amines, and cyclic amines, such as benzylamines, pyrrolidines, piperidine, morpholine, and piperazine, and inorganic salts derived from sodium, calcium, potassium, magnesium, manganese, iron, copper, zinc, aluminum, and lithium.

The invention also relates to pharmaceutically acceptable prodrugs of the compounds of the invention. The term “prodrug” means a precursor of a designated compound that, following administration to a subject, yields the compound in vivo via a chemical or physiological process such as solvolysis or enzymatic cleavage, or under physiological conditions (e.g., a prodrug on being brought to physiological pH is converted to the compound of Formula (I)). A “pharmaceutically acceptable prodrug” is a prodrug that is non-toxic, biologically tolerable, and otherwise biologically suitable for administration to the subject. Illustrative procedures for the selection and preparation of suitable prodrug derivatives are described, for example, in “Design of Prodrugs”, ed. H. Bundgaard, Elsevier, 1985.

Examples of prodrugs include compounds having an amino acid residue, or a polypeptide chain of two or more (e.g., two, three or four) amino acid residues, covalently joined through an amide or ester bond to a free amino, hydroxy, or carboxylic acid group of the compound. Examples of amino acid residues include the twenty naturally occurring amino acids, commonly designated by three letter symbols, as well as 4-hydroxyproline, hydroxylysine, demosine, isodemosine, 3-methylhistidine, norvalin, beta-alanine, gamma-aminobutyric acid, citrulline homocysteine, homoserine, ornithine and methionine sulfone.

Additional types of prodrugs may be produced, for instance, by derivatizing free carboxyl groups of structures of the compounds as amides or alkyl esters. Examples of amides include those derived from ammonia, primary C₁₋₆alkyl amines and secondary di(C₁₋₆alkyl) amines. Secondary amines include 5- or 6-membered heterocycloalkyl or heteroaryl ring moieties. Examples of amides include those that are derived from ammonia, C₁₋₃alkyl primary amines, and di(C₁₋₂alkyl)amines. Examples of esters of the invention include C₁₋₇alkyl, C₅₋₇cycloalkyl, phenyl, and phenyl(C₁₋₆alkyl) esters. Preferred esters include methyl esters. Prodrugs may also be prepared by derivatizing free hydroxy groups using groups including hemisuccinates, phosphate esters, dimethylaminoacetates, and phosphoryloxymethyloxycarbonyls, following procedures such as those outlined in Adv. Drug Delivery Rev. 1996, 19, 115. Carbamate derivatives of hydroxy and amino groups may also yield prodrugs. Carbonate derivatives, sulfonate esters, and sulfate esters of hydroxy groups may also provide prodrugs. Derivatization of hydroxy groups as (acyloxy)methyl and (acyloxy)ethyl ethers, wherein the acyl group may be an alkyl ester, optionally substituted with one or more ether, amine, or carboxylic acid functionalities, or where the acyl group is an amino acid ester as described above, is also useful to yield prodrugs. Prodrugs of this type may be prepared as described in J. Med. Chem. 1996, 39, 10. Free amines can also be derivatized as amides, sulfonamides or phosphonamides. All of these prodrug moieties may incorporate groups including ether, amine, and carboxylic acid functionalities.

The present invention also relates to pharmaceutically active metabolites of compounds of Formula (I). A “pharmaceutically active metabolite” means a pharmacologically active product of metabolism in the body of the compound or salt thereof. Prodrugs and active metabolites of a compound may be determined using routine techniques known or available in the art. See, e.g., Bertolini et al., J. Med. Chem. 1997, 40, 2011-2016; Shan et al., J. Pharm. Sci. 1997, 86 (7), 765-767; Bagshawe, Drug Dev. Res. 1995, 34, 220-230; Bodor, Adv. Drug Res. 1984, 13, 224-331; Bundgaard, Design of Prodrugs (Elsevier Press, 1985); and Larsen, Design and Application of Prodrugs, Drug Design and Development (Krogsgaard-Larsen, et al., eds., Harwood Academic Publishers, 1991).

The compounds of Formula (I) and their pharmaceutically acceptable salts, pharmaceutically acceptable prodrugs, and pharmaceutically active metabolites (collectively, “agents”) of the present invention are useful as TRPV1 modulators in the methods of the invention. The agents may be used in the inventive methods for the treatment of medical conditions, diseases, or disorders, including symptoms or disease states, mediated through modulation of TRPV1, such as those described herein.

Accordingly, the invention relates to methods of using the agents to treat subjects diagnosed with or suffering from a disease, disorder, or condition mediated through TRPV1 activity, such as: i) pain (acute, chronic, inflammatory, or neuropathic pain); ii) itch or various inflammatory disorders; iii) inner ear disorders; iv) fever or other disorders of thermoregulation; v) tracheobronchial or diaphragmatic dysfunction; vi) gastrointestinal or urinary tract disorders; or vii) disorders associated with reduced blood flow to the CNS or CNS hypoxia.

In a preferred embodiment, an agent of the present invention is administered to treat pain. Certain types of pain may be considered a disease or disorder, while other types may be considered symptoms of various diseases or disorders, and pain may include various etiologies. Exemplary types of pain treatable with a TRPV1-modulating agent according to the invention include pain associated with, arising from, or caused by: osteoarthritis, rotator cuff disorders, arthritis (e.g., rheumatoid arthritis or inflammatory arthritis; see, Barton et al. Exp. Mol. Pathol. 2006, 81(2), 166-170), fibromyalgia, migraine and headache (e.g. cluster headache, sinus headache, or tension headache; see, Goadsby Curr. Pain Headache Reports 2004, 8, 393), sinusitis, oral mucositis, toothache, dental trauma, dental extractions, dental infections, burn (Bolcskei et al., Pain 2005, 117(3), 368-376), sunburn, dermatitis, psoriasis, eczema, insect sting or bite, musculoskeletal disorders, bony fractures, ligamentous sprains, plantar fasciitis, costochondritis, tendonitis, bursitis, tennis elbow, pitcher's elbow, patellar tendonitis, repetitive strain injury, myofascial syndrome, muscle strain, myositis, temporomandibular joint disorder, amputation, low back pain, spinal cord injury, neck pain, whiplash, bladder spasms, GI tract disorders, cystitis, interstitial cystitis, cholecystitis, urinary tract infection, urethral colic, renal colic, pharyngitis, cold sores, stomatitis, external otitis, otitis media (Chan et al., Lancet 2003, 361, 385), burning mouth syndrome, mucositis, esophageal pain, esophageal spasms, abdominal disorders, gastroesophageal reflux disease, pancreatitis, enteritis, irritable bowel disorder, inflammatory bowel disease, Crohn's disease, ulcerative colitis, colon distension, abdominal constriction, diverticulosis, diverticulitis, intestinal gas, hemorrhoids, anal fissures, anorectal disorders, prostatitis, epididymitis, testicular pain, proctitis, rectal pain, labor, childbirth, endometriosis, menstrual cramps, pelvic pain, vulvodynia, vaginitis, orolabial and genital infections (e.g. herpes simplex), pleurisy, pericarditis, non-cardiac chest pain, contusions, abrasions, skin incision (Honore, P. et al., J. Pharmacol. Exp. Ther. 2005, 314, 410-21), postoperative pain, peripheral neuropathy, central neuropathy, diabetic neuropathy, acute herpetic neuralgia, post-herpetic neuralgia, trigeminal neuralgia, glossopharyngeal neuralgia, atypical facial pain, gradiculopathy, HIV associated neuropathy, physical nerve damage, causalgia, reflex sympathetic dystrophy, sciatica, cervical, thoracic or lumbar radiculopathy, brachial plexopathy, lumbar plexopathy, neurodegenerative disorders, occipital neuralgia, intercostal neuralgia, supraorbital neuralgia, inguinal neuralgia, meralgia paresthetica, genitofemoral neuralgia, carpal tunnel syndrome, Morton's neuroma, post-mastectomy syndrome, post-thoracotomy syndrome, post-polio syndrome, Guillain-Barré syndrome, Raynaud's syndrome, coronary artery spasm (Printzmetal's or variant angina), visceral hyperalgesia (Pomonis, J. D. et al. J. Pharmacol. Exp. Ther. 2003, 306, 387; Walker, K. M. et al., J. Pharmacol. Exp. Ther. 2003, 304(1), 56-62), thalamic pain, cancer (e.g. pain caused by cancer, including osteolytic sarcoma, by treatment of cancer by radiation or chemotherapy, or by nerve or bone lesions associated with cancer (see, Menendez, L. et al., Neurosci. Lett. 2005, 393 (1), 70-73; Asai, H. et al., Pain 2005, 117, 19-29), or bone destruction pain (see, Ghilardi, J. R. et al., J. Neurosci. 2005, 25, 3126-31)), infection, or metabolic disease. Additionally, the compounds may be used to treat pain indications such as visceral pain, ocular pain, thermal pain, dental pain, capsaicin-induced pain (as well as other symptomatic conditions induced by capsaicin such as cough, lachrymation, and bronchospasm).

In another preferred embodiment, inventive agents are administered to treat: itch, which may arise from various sources, such as dermatological or inflammatory disorders; or inflammatory disorders selected from the group consisting of: renal or hepatobiliary disorders, immunological disorders, medication reactions and unknown/idiopathic conditions. Inflammatory disorders treatable with an inventive agent include, for example, inflammatory bowel disease (IBD), Crohn's disease, and ulcerative colitis (Geppetti, P. et al., Br. J. Pharmacol. 2004, 141, 1313-20; Yiangou, Y. et al., Lancet 2001, 357, 1338-39; Kimball, E. S. et al., Neurogastroenterol. Motil., 2004, 16, 811), osteoarthritis (Szabo, A. et al., J. Pharmacol. Exp. Ther. 2005, 314, 111-119), psoriasis, psoriatic arthritis, rheumatoid arthritis, myasthenia gravis, multiple sclerosis, scleroderma, glomerulonephritis, pancreatitis, inflammatory hepatitis, asthma, chronic obstructive pulmonary disease, allergic rhinitis, uveitis, and cardiovascular manifestations of inflammation including atherosclerosis, myocarditis, pericarditis, and vasculitis.

In another preferred embodiment, inner ear disorders are treated with an inventive agent. Such disorders include, for example, hyperacusis, tinnitus, vestibular hypersensitivity, and episodic vertigo.

In another preferred embodiment, tracheobronchial and diaphragmatic dysfunctions are treated with an inventive agent, including, for example, asthma and allergy-related immune responses (Agopyan, N. et al., Am. J. Physiol. Lung Cell Mol. Physiol. 2004, 286, L563-72; Agopyan, N. et al., Toxicol. Appl. Pharmacol. 2003, 192, 21-35), cough (e.g., acute or chronic cough, or cough caused by irritation from gastroesophageal reflux disease; see, Lalloo, U. G. et al., J. Appl. Physiol. 1995, 79(4), 1082-7), bronchospasm, chronic obstructive pulmonary disease, chronic bronchitis, emphysema, and hiccups (hiccoughs, singultus).

In yet another preferred embodiment, gastrointestinal and urinary tract disorders are treated with an inventive agent, such as, bladder overactivity, inflammatory hyperalgesia, visceral hyperreflexia of the urinary bladder, hemorrhagic cystitis (Dinis, P. et al., J. Neurosci. 2004, 24, 11253-11263), interstitial cystitis (Sculptoreanu, A. et al., Neurosci. Lett. 2005, 381, 42-46), inflammatory prostate disease, prostatitis (Sanchez, M. et al., Eur. J. Pharmacol. 2005, 515, 20-27), nausea, vomiting, intestinal cramping, intestinal bloating, bladder spasms, urinary urgency, defecation urgency and urge incontinence.

In another preferred embodiment, disorders associated with reduced blood flow to the CNS or CNS hypoxia are treated with an inventive agent. Such disorders include, for example, head trauma, spinal injury, thromboembolic or hemorrhagic stroke, transient ischaemic attacks, cerebral vasospasm, hypoglycaemia, cardiac arrest, status epilepticus, perinatal asphyxia, Alzheimer's disease, and Huntington's Disease.

In other embodiments, inventive agents are administered to treat other diseases, disorders, or conditions mediated through TRPV1 activity, such as: anxiety; learning or memory disorders; eye-related disorders (such as glaucoma, vision loss, increased intraocular pressure, and conjunctivitis); baldness (e.g., by stimulating hair growth); diabetes (including insulin-resistant diabetes or diabetic conditions mediated by insulin sensitivity or secretion); obesity (e.g., through appetite suppression); dyspepsia; biliary colic; renal colic; painful bladder syndrome; inflamed esophagus; upper airway disease; urinary incontinence; acute cystitis; and envenomations (such as marine, snake, or insect stings or bites, including jellyfish, spider, or stingray envenomations).

In especially preferred embodiments of the therapeutic methods of the invention, effective amounts of the TRPV1 modulators of the present invention are administered to treat pain, arthritis, itch, cough, asthma, or inflammatory bowel disease.

The term “treat” or “treating” as used herein is intended to refer to administration of an inventive agent or composition of matter of the invention to a subject to effect a therapeutic or prophylactic benefit through modulation of TRPV1 activity. Treating includes reversing, ameliorating, alleviating, inhibiting the progress of, lessening the severity of, or preventing a disease, disorder, or condition (or one or more symptoms of such disease, disorder or condition) mediated through modulation of TRPV1 activity. The term “subject” refers to a mammalian patient in need of such treatment, such as a human. “Modulators” include both inhibitors and activators, where “inhibitors” refer to compounds that decrease, prevent, inactivate, desensitize or down-regulate TRPV1 expression or activity, and “activators” are compounds that increase, activate, facilitate, sensitize, or up-regulate TRPV1 expression or activity.

In treatment methods according to the invention, an effective amount of at least one agent according to the invention is administered to a subject suffering from or diagnosed as having such a disease, disorder, or condition. An “effective amount” means an amount or dose generally sufficient to bring about the desired therapeutic or prophylactic benefit in patients in need of such treatment for the designated disease, disorder, or condition. Effective amounts or doses of the agents of the present invention may be ascertained by routine methods such as modeling, dose escalation studies, or clinical trials, and by taking into consideration routine factors, e.g., the mode or route of administration or drug delivery, the pharmacokinetics of the agent, the severity and course of the disease, disorder, or condition, the subject's previous or ongoing therapy, the subject's health status, and response to drugs, and the judgment of the treating physician. An exemplary dose is in the range of from about 0.001 to about 200 mg of inventive agent per kg of subject's body weight per day, preferably about 0.05 to 100 mg/kg/day, or about 1 to 35 mg/kg/day, or about 0.1 to, 10 mg/kg daily in single or divided dosage units (e.g., BID, TID, or QID). For a 70-kg human, an illustrative range for a suitable dosage amount is from about 0.05 to about 7 g/day, or about 0.2 to about 2.5 g/day. Once improvement of the patient's disease, disorder, or condition has occurred, the dose may be adjusted for preventative or maintenance treatment. For example, the dosage or the frequency of administration, or both, may be reduced as a function of the symptoms, to a level at which the desired therapeutic or prophylactic effect is maintained. Of course, if symptoms have been alleviated to an appropriate level, treatment may cease. Patients may, however, require intermittent treatment on a long-term basis upon any recurrence of symptoms.

In addition, the pharmaceutical agents of the invention may be used in combination with additional active ingredients in the treatment methods described above. The additional active ingredients may be coadministered separately with an inventive agent or included with such an agent in a pharmaceutical composition according to the invention. In an exemplary embodiment, additional active ingredients are those that are known or discovered to be effective in the treatment of conditions, disorders, or diseases mediated by TRPV1 activity, such as another TRPV1 modulator or a compound active against another target associated with the particular condition, disorder, or disease. The combination may serve to increase efficacy (e.g., by including in the combination a compound potentiating the potency or effectiveness of an agent according to the invention), decrease one or more side effects, or decrease the required dose of the agent according to the invention. In one illustrative embodiment, a composition for treating pain according to the invention may contain one or more additional active ingredients selected from opioids, NSAIDs (e.g., ibuprofen, cyclooxygenase-2 (COX-2) inhibitors, and naproxen), gabapentin, pregabalin, tramadol, acetaminophen, aspirin, and alpha-2 adrenergic agonists (e.g., brimonidine, clonidine, dexmedetomidine, mivazerol, guanabenz, guanfacine, or methyldopa).

The agents of the invention are used, alone or in combination with one or more other active ingredients, to formulate pharmaceutical compositions of the invention. A pharmaceutical composition of the invention comprises: (a) an effective amount of a pharmaceutical agent in accordance with the invention; and (b) a pharmaceutically acceptable excipient.

A “pharmaceutically acceptable excipient” refers to a substance that is non-toxic, biologically tolerable, and otherwise biologically suitable for administration to a subject, such as an inert substance, added to a pharmacological composition or otherwise used as a vehicle, carrier, or diluent to facilitate administration of an inventive agent and that is compatible therewith. Examples of excipients include calcium carbonate, calcium phosphate, various sugars and types of starch, cellulose derivatives, gelatin, vegetable oils, and polyethylene glycols.

Delivery forms of the pharmaceutical compositions containing one or more dosage units of the pharmaceutical agents may be prepared using suitable pharmaceutical excipients and compounding techniques known or that become available to those skilled in the art. The compositions may be administered in the inventive methods by a suitable route of delivery, e.g., oral, parenteral, rectal, topical, or ocular routes, or by inhalation.

The preparation may be in the form of tablets, capsules, sachets, dragees, powders, granules, lozenges, powders for reconstitution, liquid preparations, or suppositories. Preferably, the compositions are formulated for intravenous infusion, topical administration, or oral administration.

For oral administration, the compounds of the invention can be provided in the form of tablets or capsules, or as a solution, emulsion, or suspension. To prepare the oral compositions, the agents may be formulated to yield a dosage of, e.g., from about 0.05 to about 50 mg/kg daily, or from about 0.05 to about 20 mg/kg daily, or from about 0.1 to about 10 mg/kg daily.

Oral tablets may include the inventive agent and any other active ingredients mixed with compatible pharmaceutically acceptable excipients such as diluents, disintegrators, binders, lubricants, sweeteners, flavors, colors, and preservatives. Suitable inert fillers include sodium and calcium carbonate, sodium and calcium phosphate, lactose, starch, sugar, glucose, methyl cellulose, magnesium stearate, mannitol, sorbitol, and the like. Exemplary liquid oral excipients include ethanol, glycerol, water, and the like. Starch, polyvinyl-pyrrolidone (PVP), sodium starch glycolate, microcrystalline cellulose, and alginic acid are exemplary disintegrators. Binders may include starch and gelatin. The lubricator, if present, may be magnesium stearate, stearic acid or talc. If desired, the tablets may be coated with a material such as glyceryl monostearate or glyceryl distearate to delay absorption in the gastrointestinal tract, or may be coated with an enteric coating.

Capsules for oral administration include hard and soft gelatin capsules. To prepare hard gelatin capsules, the inventive agent may be mixed with a solid, semi-solid, or liquid diluent. Soft gelatin capsules may be prepared by mixing the inventive agent with water, an oil such as peanut oil, sesame oil, or olive oil, liquid paraffin, a mixture of mono and di-glycerides of short chain fatty acids, polyethylene glycol 400, or propylene glycol.

Liquids for oral administration may be in the form of suspensions, solutions, emulsions or syrups or may be lyophilized or presented as a dry product for reconstitution with water or other suitable vehicle before use. Such liquid compositions may optionally contain: pharmaceutically-acceptable excipients such as suspenders (for example, sorbitol, methyl cellulose, sodium alginate, gelatin, hydroxyethylcellulose, carboxymethylcellulose, aluminum stearate gel and the like); non-aqueous vehicles, e.g., oil (for example, almond oil or fractionated coconut oil), propylene glycol, ethyl alcohol, or water; preservatives (for example, methyl or propyl p-hydroxybenzoate or sorbic acid); wetting agents such as lecithin; and, if desired, flavoring or coloring agents.

The agents of this invention may also be administered by non-oral routes. For example, compositions may be formulated for rectal administration as a suppository. For parenteral use, including intravenous, intramuscular, intraperitoneal, or subcutaneous routes, the agents of the invention may be provided in sterile aqueous solutions or suspensions, buffered to an appropriate pH and isotonicity or in parenterally acceptable oil. Suitable aqueous vehicles include Ringer's solution and isotonic sodium chloride. Such forms may be presented in unit-dose form such as ampules or disposable injection devices, in multi-dose forms such as vials from which the appropriate dose may be withdrawn, or in a solid form or pre-concentrate that can be used to prepare an injectable formulation. Illustrative infusion doses range from about 1 to 1000 μg/kg/minute of agent admixed with a pharmaceutical carrier over a period ranging from several minutes to several days.

For topical administration, the agents may be mixed with a pharmaceutical carrier at a concentration of about 0.1% to about 10% of drug to vehicle. Another mode of administering the agents of the invention may utilize a patch formulation to effect transdermal delivery.

Inventive agents may alternatively be administered in methods of this invention by inhalation, via the nasal or oral routes, e.g., in a spray formulation also containing a suitable carrier.

Exemplary chemical entities useful in methods of the invention will now be described by reference to illustrative synthetic schemes for their general preparation below and the specific examples that follow. Artisans will recognize that, to obtain the various compounds herein, starting materials may be suitably selected so that the ultimately desired substituents will be carried through the reaction scheme with or without protection as appropriate to yield the desired product. Alternatively, it may be necessary or desirable to employ, in the place of the ultimately desired substituent, a suitable group that may be carried through the reaction scheme and replaced as appropriate with the desired substituent. Unless otherwise specified, the variables in the formulas depicted in the schemes below are as defined above in reference to Formula (I).

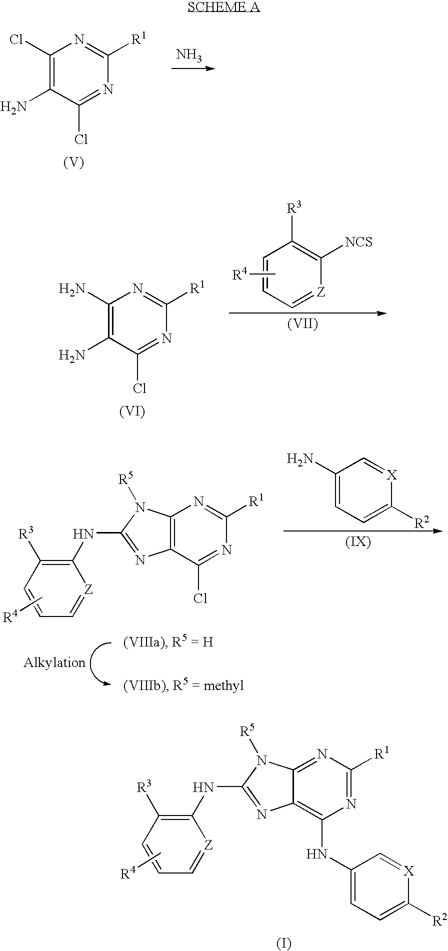

The present invention also contemplates methods of making compounds of Formula (I), and pharmaceutically acceptable salts thereof, as shown in general Scheme A, and chemical intermediates of formula (VIII), which are useful in the processes of the invention. The method of making a compound of Formula (I) comprises reacting a compound of formula (VIII) (which includes (VIIIa) and (VIIIb)) with an aromatic amine (IX) to provide a compound of Formula (I). In preferred embodiments, reactions are performed in the presence of an acid catalyst, preferably p-toluenesulfonic acid, methanesulfonic acid, HCl, or trifluoroacetic acid (TFA), in a solvent such as toluene, dioxane, acetonitrile, isopropanol, water, or a mixture thereof, at a temperature from about 70 to about 150° C., optionally using microwave irradiation or a sealed tube. Preferred conditions involve treatment of a chloro-pyrimidine (VIII) with an aromatic amine (IX) and HCl in isopropanol at reflux temperature. Alternatively, reaction of compounds (VIIIa) or (VIIIb) with aromatic amines (IX) is accomplished under palladium coupling conditions, in the presence of a palladium (0) catalyst (used directly or formed in situ), a phosphine ligand (such as PPh₃, (tBu)₃P, (cyclohexyl)₃P, 1,1′-bis(diphenylphosphino)ferrocene, 1,2,3,4,5-pentaphenyl-1-(di-t-butylphosphino)ferrocene, or 2-(dicyclohexylphosphino)biphenyl), and a base (such as NaOtBu, KOtBu, K₃PO₄, KOH, K₂CO₃, Cs₂CO₃, Et₃N, NaOH, Na₃PO₄, Na₂CO₃, or a mixture thereof), in a polar organic solvent (such as acetonitrile, toluene, DMF, ethylene glycol dimethyl ether (DME), tetrahydrofuran (THF), methanol (MeOH), EtOH, water, or a mixture thereof). Palladium coupling reactions are generally performed at temperatures from about room temperature to the reflux temperature of the solvent.

The method of making a compound of Formula (I) further comprises reacting a compound of formula (VI) with an isothiocyanate (VII) to form a compound (VIIIa). In preferred embodiments, reactions are performed in the presence of a suitable base, such as iPr₂NEt, Et₃N, 1,8-diazabicyclo[5.4.0]undec-7-ene (DBU) or Cs₂CO₃, in a solvent such as acetonitrile, at a temperature from about room temperature (rt) to about 100° C. Exemplary conditions include treatment with iPr₂NEt in acetonitrile at about 90 IC in a sealed tube. The method optionally further comprises alkylation of an amine (VIIIa) with a methyl halide reagent, such as MeI, the presence of a suitable base such as K₂CO₃, Na₂CO₃, or Et₃N, in a solvent such as N,N-dimethylformamide (DMF) or THF, to give a compound (VIIIb).

The method of making a compound of Formula (I) further comprises reacting a dichloro-pyrimidine (V), which is commercially available or may be prepared according to known general processes, with ammonia or an ammonia equivalent (such as NH₄OAc), in a solvent such as methanol (MeOH), at a temperature from about 50° C. to about 100° C., using a microwave reactor or a sealed tube, to give a diaminopyrimidine compound of formula (VI).

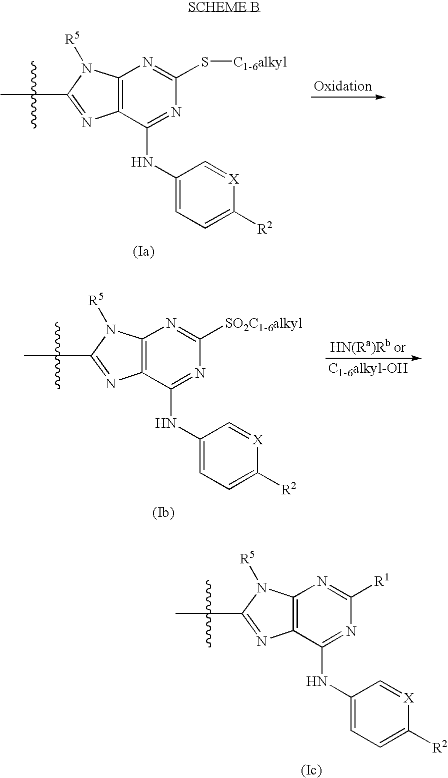

As shown in general Scheme B, embodiments of Formula (I) (shown in abbreviated form) where R¹ is —S—C₁₋₆alkyl (la) may be converted into other compounds of Formula (I), such as (Ib) and (Ic). Oxidation of thioethers (Ia) yields sulfones (Ib), and may be accomplished by reaction with a suitable oxidizing agent such as KHSO₅, meta-chloroperbenzoic acid (mCPBA), or dimethyldioxirane, in a solvent such as CH₂Cl₂, MeOH, tetrahydrofuran (THF), water, or a mixture thereof. Exemplary conditions include treatment with KHSO₅ (about 3 equivalents) in MeOH/THF/water at about 40° C. Displacement of the sulfone substituent to obtain a compound of formula (Ic) where R¹ is —O—C₁₋₆alkyl is attained by reaction with the corresponding alcohol, optionally used as the solvent, in the presence of a suitable base, such as NaH, KOtBu, or NaO—C₁₋₆alkyl, at a temperature between about room temperature and about 100° C., optionally using a sealed tube. For example, where R¹ is —OCH₃, preferred conditions conditions include heating with NaOMe in MeOH at 80° C. in a sealed tube. Displacement of the sulfone substituent with amines HN(R^{a})R^{b} yields compounds of formula (Ic) where R¹ is —NR^{a}R^{b}, and may be performed neat or in alcoholic solvents such as MeOH, ethanol (EtOH), tBuOH, n-BuOH, t-amyl-OH, or a mixture thereof, or in a solvent such as toluene or benzene, at temperatures from about room temperature to about 150° C., optionally using a sealed tube. In preferred embodiments, reactions are run in t-amyl-OH at a temperature of about 130° C. in a sealed tube.

Compounds of Formula (I) may be converted to their corresponding salts using general methods described in the art. For example, amines of Formula (I) may be treated with trifluoroacetic acid, HCl, sulfuric acid, phosphoric-acid, or citric acid in a solvent such as diethyl ether (Et₂O), CH₂Cl₂, THF, MeOH, or isopropanol to provide the corresponding salt forms.

Compounds prepared according to the schemes described above may be obtained as single enantiomers, diastereomers, or regioisomers, by enantio-, diastero-, or regiospecific synthesis, or by resolution. Compounds prepared according to the schemes above may alternately be obtained as racemic (1:1) or non-racemic (not 1:1) mixtures or as mixtures of diastereomers or regioisomers. Where racemic and non-racemic mixtures of enantiomers are obtained, single enantiomers may be isolated using conventional separation techniques, such as chiral chromatography, recrystallization, diastereomeric salt formation, derivatization into diastereomeric adducts, biotransformation, or enzymatic transformation. Where regioisomeric or diastereomeric mixtures are obtained, single isomers may be separated using known techniques such as chromatography or crystallization.

The following specific examples are provided to illustrate various preferred embodiments of pharmaceutical agents according to the invention.

### EXAMPLES

### Chemistry

In the examples below, the following experimental and analytical protocols were followed unless otherwise indicated.

Where solutions were “concentrated”, they were concentrated using a rotary evaporator under reduced pressure. Unless otherwise specified, reaction solutions were stirred at room temperature (rt) under a N_{2(g)} atmosphere.

Microwave reactions were carried out in either a CEM Discover® or a Biotage Initiator™ Microwave at specified temperatures.

Where solutions were dried, they were dried over MgSO₄ or Na₂SO₄.

Normal phase purification was typically done by normal phase flash column chromatography (FCC) with RediSep® silica gel columns using ethyl acetate (EtOAc)/hexanes as eluent unless otherwise specified.

Preparative Reversed-Phase high performance liquid chromatography (HPLC) was performed on a Shimadzu® instrument with a Phenomenex Gemini column (C18; 5 μm, 150×21.2 mm) or Waters Xterra RP18 OBD column (5 μm, 100×30 mm), a flow rate of 30 mL/min (Gemini) or 80 mL/min (Waters), detection at λ=254 nm. The eluent was 0.05% TFA in an acetonitrile/H₂O gradient, ramped over 20 min.

Unless otherwise indicated, Example compounds were obtained as free bases following FCC or as trifluoroacetic acid salts following reverse phase HPLC purification.

NMR spectra were obtained on Bruker model DRX spectrometers. The format of ¹H NMR data below is: chemical shift in ppm downfield of the tetramethylsilane reference (multiplicity, coupling constant J in Hz, integration).

Mass spectra were obtained on an Agilent series 1100 MSD using electrospray ionization (ESI) in either positive or negative modes as indicated. Calculated mass corresponds to the exact mass.

Chemical names were generated using ChemDraw Ultra 6.0.2 (CambridgeSoft Corp., Cambridge, Mass.) or ACD/Name Version 9 (Advanced Chemistry Development, Toronto, Ontario, Canada).

### Intermediate 1: 6-Chloro-pyrimidine-4,5-diamine

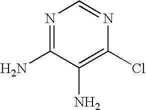

To a 7 N solution of ammonia in MeOH (40 mL) was added 4,6-dichloro-pyrimidin-5-ylamine (8.7 g, 53 mmol) and the solution was heated to 100° C. in a sealed tube. After 12 h, the resulting solution was cooled to rt and allowed to stand for 2 h. The colorless crystalline material that resulted was collected by filtration and washed with ice cold MeOH (10 mL). MS (ESI): mass calcd. for C₄H₅ClN₄, 144.0; m/z found, 145.0 [M+H]⁺. ¹H NMR ((CD₃)₂SO): 7.64 (s, 1H), 6.70 (s, 2H), 4.93 (s, 2H).

### Intermediate 2: 6-Chloro-2-methyl-pyrimidine-4,5-diamine

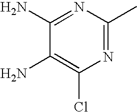

The title compound was prepared using a method analogous to that described for Intermediate 1. MS (ESI): mass calcd. for C₅H₇ClN₄, 158.0; m/z found, 159.0 [M+H]⁺. ¹H NMR ((CD₃)₂SO): 6.65 (s, 2H), 4.71 (s, 2H), 2.19 (s, 3H).

### Intermediate 3: (6-Chloro-9H-purin-8-yl)-(2,6-dichloro-phenyl)-amine.

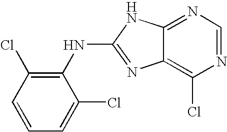

To a mixture of 6-chloro-pyrimidine-4,5-diamine (188 mg, 1.31 mmol), 1,3-dichloro-2-isothiocyanato-benzene (266 mg, 1.31 mmol), and CH₃CN (5 mL) was added iPr₂NEt (337 mg, 2.62 mmol) at rt. The mixture was heated to 90° C. in a sealed tube. After 12 h, the reaction was cooled and purified by preparative reverse-phase HPLC to afford the title compound as a colorless solid (80 mg, 20%). MS (ESI): mass calcd. for C₁₁H₆Cl₃N₅, 312.9; m/z found, 314.0 [M+H]⁺. ¹H NMR ((CD₃)₂SO): 9.90 (br s, 1H), 8.39 (s, 1H), 7.62 (d, J=8.1 Hz, 2H), 7.43 (t, J=8.1 Hz, 1H).

### Intermediate 4: (6-Chloro-9-methyl-9H-purin-8-yl)-(2,6-dichloro-phenyl)-amine

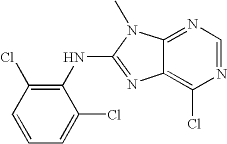

To a mixture (6-chloro-9H-purin-8-yl)-(2,6-dichloro-phenyl)-amine (100 mg, 0.32 mmol) and K₂CO₃ (88 mg, 0.64 mmol) in DMF (2 mL) was added MeI (20 uL, 0.32 mmol) at rt. After 2 h, the resulting mixture was partioned between H₂O (15 mL) and EtOAc (10 mL). The layers were separated and the aqueous layer was extracted with EtOAc (2×20 mL). The combined organic extracts were dried (MgSO₄), filtered and concentrated. The residue was purified by preparative reverse-phase HPLC to afford the title compound as a colorless solid (40 mg, 38%). MS (ESI): mass calcd. for C₁₂H₈Cl₃N₅, 326.9; m/z found, 328.0 [M+H]⁺. ¹H NMR (CD₃OD): 8.46 (s, 1H), 7.56 (d, J=8.1 Hz, 2H), 7.39 (dd, J=8.5, 7.8 Hz, 1H), 3.83 (s, 3H).

Intermediates 5-8 were prepared using methods analogous to those described for Intermediate 3.

### Intermediate 5: (6-Chloro-2-methyl-9H-purin-8-yl)-(2,6-dichloro-phenyl)-amine.

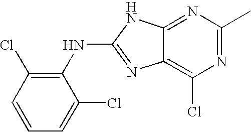

MS (ESI): mass calcd. for C₁₂H₈Cl₃N₅, 326.9; m/z found, 328.0 [M+H]⁺. ¹H NMR ((CD₃)₂SO): 7.61 (d, J=8.1 Hz, 2H), 7.41 (t, J=8.1 Hz, 1H), 2.52 (s, 3H).

### Intermediate 6: (2-Chloro-phenyl)-(6-chloro-9H-purin-8-yl)-amine

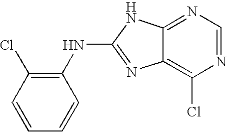

MS (ESI): mass calcd. for C₁₁H₇Cl₂N₅, 279.0; m/z found, 280.0 [M+H]⁺.

### Intermediate 7: (6-Chloro-9H-purin-8-yl)-(2-trifluoromethyl-Phenyl)-amine

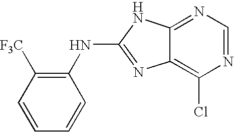

MS (ESI): mass calcd. for C₁₂H₇Cl₂F₃N₅, 313.0; m/z found, 314.0 [M+H]⁺. ¹H NMR ((CD₃)₂SO): 9.35 (br s, 1H), 8.44 (s, 1H), 8.07 (d, J=8.0 Hz, 1H), 7.82-7.74 (m, 2H), 7.46 (t, J=7.7 Hz, 1H).

### Example 1

### N⁸-(2,6-Dichloro-phenyl)-N⁶-(4-trifluoromethyl-phenyl)-9H-purine-6,8-diamine

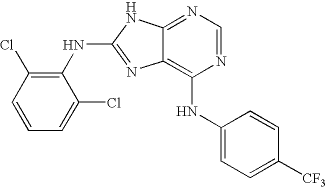

To a solution of (6-chloro-9H-purin-8-yl)-(2,6-dichloro-phenyl)-amine (212 mg, 0.67 mmol) and 4-trifluoromethyl-phenylamine (109 mg, 0.67 mmol) in isopropyl alcohol (IPA) (3 mL) was added HCl (1.25 M in IPA; 1.49 mmol, 1.20 mL). The resulting solution was heated in a sealed tube to 90° C. After 12 h, the solution was cooled and purified by preparative reverse-phase HPLC to afford the title compound as a colorless solid (200 mg, 67%). MS (ESI): mass calcd. for C₁₈H₁₁Cl₂F₃N₆, 438.0; m/z found, 439.1 [M+H]⁺. ¹H NMR ((CD₃)₂SO): 9.75 (s, 1H), 8.46 (s, 1H), 7.96 (d, J=8.5 Hz, 2H), 7.73-7.62 (m, 4H), 7.43 (t, J=8.1 Hz, 1H).

The compounds in Examples 2-10 were prepared using methods analogous to those described for Example 1.

### Example 2

### N⁸-(2,6-Dichloro-phenyl)-N⁶-(6-trifluoromethyl-pyridin-3-yl)-9H-purine-6,8-diamine

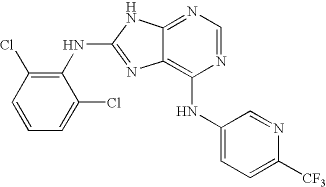

MS (ESI): mass calcd. for C₁₇H₁₀Cl₂F₃N₇, 439.0; m/z found, 440.0 [M+H]⁺. ¹H NMR ((CD₃)₂SO): 9.90 (br s, 1H), 9.04 (d, J=1.2 Hz, 1H), 8.59-8.55 (m, 1H), 8.44-8.39 (m, 1H), 7.89-7.84 (m, 1H), 7.65 (d, J=7.4 Hz, 2H), 7.47-7.39 (m, 1H).

### Example 3

### N⁶-(4-tert-Butyl-phenyl)-N⁸-(2,6-dichloro-phenyl)-9H-purine-6,8-diamine

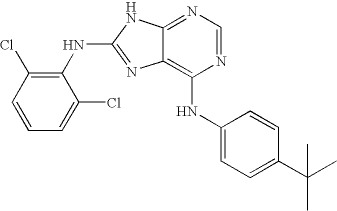

MS (ESI): mass calcd. for C₂₁H₂₀Cl₂N₆, 426.1; m/z found, 427.1 [M+H]⁺. ¹H NMR ((CD₃)₂SO): 9.59 (br s, 1H), 8.44 (s, 1H), 7.67 (d, J=8.1 Hz, 2H), 7.60-7.56 (m, 2H), 7.45 (t, J=8.1 Hz, 1H), 7.39 (d, J=8.6 Hz, 2H), 1.29 (s, 9H).

### Example 4

### N⁸-(2,6-Dichloro-phenyl)-N⁶-phenyl-9H-purine-6,8-diamine

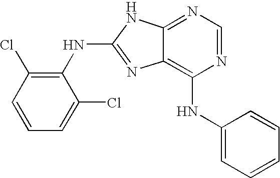

MS (ESI): mass calcd. for C₁₇H₁₂Cl₂N₆, 370.0; m/z found, 371.1 [M+H]⁺. ¹H NMR ((CD₃)₂SO): 9.66 (br s, 1H), 8.50-8.47 (m, 1H), 7.71-7.65 (m, 5H), 7.48-7.44 (m, 1H), 7.39 (t, J=7.6 Hz, 3H), 7.15-7.10 (m, 1H).

### Example 5

### N⁸-(2,6-Dichloro-phenyl)-N⁶-(4-methanesulfonyl-phenyl)-9H-purine-6,8-diamine

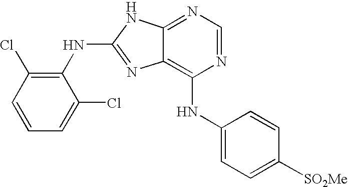

MS (ESI): mass calcd. for C₁₈H₁₄Cl₂N₆O₂S, 448.0; m/z found, 449.0 [M+H]⁺. ¹H NMR ((CD₃)₂SO): 9.79 (br s, 1H), 8.45 (br s, 1H), 8.01 (d, J=8.3 Hz, 2H), 7.87 (d, J=8.5 Hz, 2H), 7.66 (d, J=8.1 Hz, 2H), 7.47-7.40 (m, 1H), 3.18 (s, 3H).

### Example 6

### N⁸-(2,6-Dichloro-phenyl)-2-methyl-N⁶-(4-trifluoromethyl-phenyl)-9H-purine-6,8-diamine

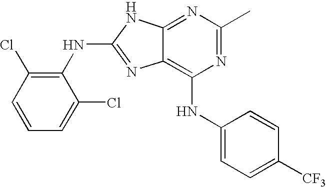

MS (ESI): mass calcd. for C₁₉H₁₃Cl₂F₃N₆, 452.0; m/z found, 453.1 [M+H]⁺. ¹H NMR ((CD₃)₂SO): 10.55 (br s, 1H), 10.10 (br s, 1H), 7.93 (d, J=8.5 Hz, 2H), 7.74 (d, J=8.6 Hz, 2H), 7.67 (d, J=8.2 Hz, 2H), 7.47 (t, J=8.2 Hz, 1H), 2.57 (s, 3H).

### Example 7

### N⁸-(2-Chloro-phenyl)-N⁶-(4-trifluoromethyl-phenyl)-9H-purine-6,8-diamine

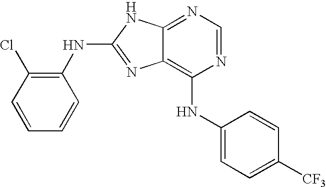

MS (ESI): mass calcd. for C₁₈H₁₂ClF₃N₆, 404.0; m/z found, 405.1 [M+H]⁺. ¹H NMR ((CD₃)₂SO): 9.90 (br s, 1H), 8.49-8.45 (m, 2H), 8.06-8.00 (m, 2H), 7.72 (d, J=8.6 Hz, 2H), 7.58-7.55 (m, 1H), 7.45-7.40 (m, 1H) 7.20-7.15 (m, 1H).

### Example 8

### N⁶-(4-Trifluoromethyl-phenyl)-N⁸-(2-trifluoromethyl-phenyl)-9H-purine-6,8-diamine

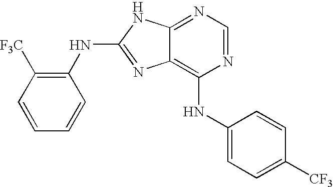

MS (ESI): mass calcd. for, C₁₉H₁₂F₆N₆, 438.1; m/z found, 439.1 [M+H]⁺. ¹H NMR (CD₃OD): 8.52 (s, 1H), 7.97-7.91 (m, 3H), 7.87 (d, J=7.9 Hz, 1H), 7.82-7.78 (m, 1H), 7.69 (d, J=8.7 Hz, 2H), 7.57 (t, J=7.7 Hz, 1H).

### Example 9

### N⁸-(2-Trifluoromethyl-phenyl)-N⁶-(6-trifluoromethyl-pyridin-3-yl)-9H-purine-6,8-diamine

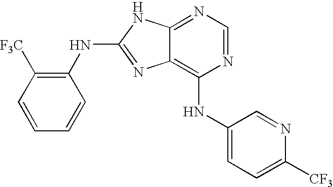

MS (ESI): mass calcd. for C₁₈H₁₁F₆N₇, 439.1; m/z found, 440.1 [M+H]⁺. ¹H NMR ((CD₃)₂SO): 10.05-10.03 (m, 1H), 9.06 (s, 1H), 8.58-8.54 (m, 1H), 8.46 (s, 1H), 8.21-8.17 (m, 1H), 7.88 (d, J=8.7 Hz, 1H), 7.83-7.73 (m, 2H), 7.46-7.41 (m, 1H).

### Example 10

### N⁸-(2,6-Dichloro-phenyl)-9-methyl-N⁶-(4-trifluoromethyl-phenyl)-9H-purine-6,8-diamine

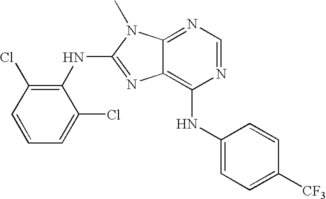

MS (ESI): mass calcd. for C₁₉H₁₃Cl₂F₃N₆, 452.0; m/z found, 453.1 [M+H]⁺. ¹H NMR ((CD₃)₂SO): 9.59 (br s, 1H), 8.32 (s, 1H), 8.04 (d, J=8.1 Hz, 2H), 7.63-7.57 (m, 4H), 7.34-7.28 (m, 1H), 3.70 (s, 3H).

The compounds in Examples 11-13 may be prepared using methods analogous to those described for Example 1.

### Example 11

### N⁶-(4-Trifluoromethyl-phenyl)-N⁸-(3-trifluoromethyl-pyridin-2-yl)-9H-purine-6,8-diamine

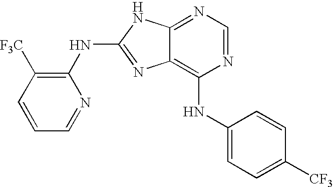

### Example 12

### N⁸-(2,6-Dichloro-phenyl)-2-methylsulfanyl-N⁶-(4-trifluoromethyl-phenyl)-9H-purine-6,8-diamine

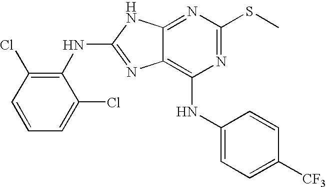

### Example 13

### N⁸-(2,6-Dichloro-phenyl)-2-methoxy-N⁶-(4-trifluoromethyl-phenyl)-9H-purine-6,8-diamine

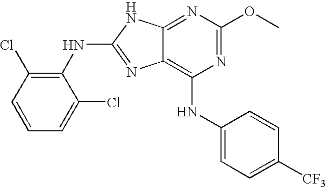

### Example 14

### N⁸-(2,6-Dichloro-phenyl)-2-methanesulfonyl-N⁶-(4-trifluoromethyl-phenyl)-9H-purine-6,8-diamine

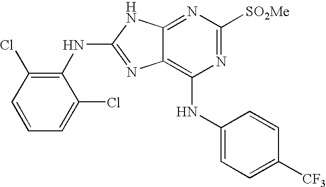

To a solution of N⁸-(2,6-dichloro-phenyl)-2-methylsulfanyl-W-(4-trifluoromethyl-phenyl)-9H-purine-6,8-diamine (1 equiv.) in 1:1 THF/MeOH (˜0.02 M total) is added potassium peroxymonosulfate (˜0.1 M solution in H₂O; 3 equiv.). The resulting mixture is stirred vigorously at −40° C. After 24 h, the mixture is concentrated and the crude residue is diluted with satd. aq. NaHCO₃ and extracted with EtOAc. The combined organic layers are dried, concentrated, and purified by FCC to afford the title compound.

### Example 15

### N⁸-(2,6-Dichloro-phenyl)-2-morpholin-4-yl-N⁶-(4-trifluoromethyl-phenyl)-9H-purine-6,8-diamine

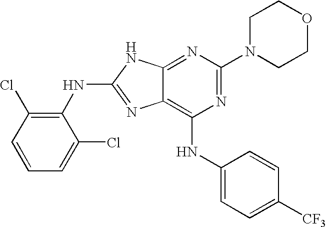

To a mixture of N⁸-(2,6-dichloro-phenyl)-2-methanesulfonyl-N⁶-(4-trifluoromethyl-phenyl)-9H-purine-6,8-diamine (1 equiv.) in tert-amyl alcohol (˜0.05 M) is added morpholine (3 equiv.). The resulting mixture is heated to 130° C. in a sealed tube. After 12 h, the solution is cooled and purified by preparative reverse-phase HPLC to afford the title compound.

The compounds in Examples 16-21 may be prepared using methods analogous to those described for Example 15.

### Example 16

### N⁸-(2,6-Dichloro-phenyl)-2-(4-methyl-piperazin-1-yl)-N⁶-(4-trifluoromethyl-Phenyl)-9H-purine-6,8-diamine

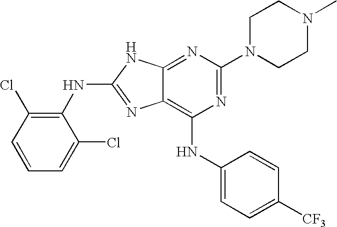

### Example 17

### N⁸-(2,6-Dichloro-phenyl)-2-(4-isobutyl-piperazin-1-yl)-N⁶-(4-trifluoromethyl-Phenyl)-9H-purine-6,8-diamine

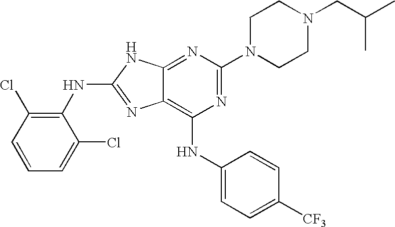

### Example 18

### N⁸-(2,6-Dichloro-phenyl)-2-piperidin-1-yl-N⁶-(4-trifluoromethyl-phenyl)-9H-purine-6,8-diamine

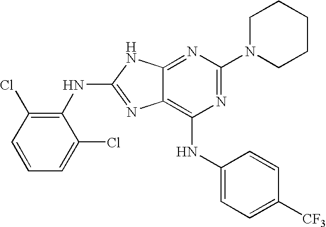

### Example 19

### N⁸-(2,6-Dichloro-phenyl)-N²-isobutyl-N⁶-(4-trifluoromethyl-phenyl)-9H-purine-2,6,8-triamine

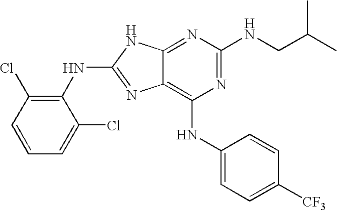

### Example 20

### N⁸-(2,6-Dichloro-phenyl)-N²-isopropyl-N⁶-(4-trifluoromethyl-phenyl)-9H-purine-2,6,8-triamine

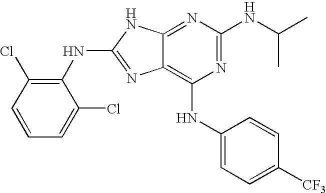

### Example 21

### N⁸-(2,6-Dichloro-phenyl)-2-pyrrolidin-1-yl-N⁶-(4-trifluoromethyl-phenyl)-9H-purine-6,8-diamine

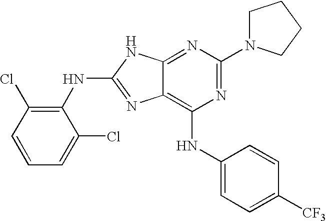

The compounds in Examples 22-26 may be prepared using methods analogous to those described in the preceding examples.

### Example 22

### N⁸-(2,6-Dichloro-phenyl)-N⁶-(3-fluoro-4-trifluoromethyl-phenyl)-9H-purine-6,8-diamine

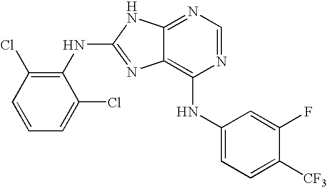

### Example 23

### N⁶-(3-Chloro-4-trifluoromethyl-phenyl)-N⁸-(2,6-dichloro-phenyl)-9H-purine-6,8-diamine

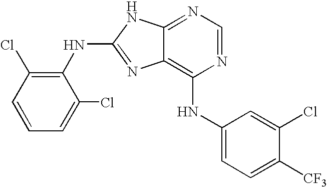

### Example 24

### N⁸-(2,6-Dichloro-phenyl)-N⁶-(3-fluoro-4-methanesulfonyl-phenyl)-9H-purine-6,8-diamine

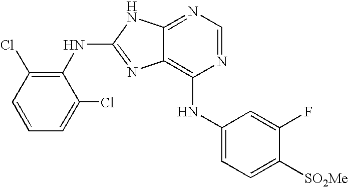

### Example 25

### N⁸-(2,6-Dichloro-phenyl)-N²-pyridin-2-ylmethyl-N⁶-(4-trifluoromethyl-phenyl)-9H-purine-2,6,8-triamine

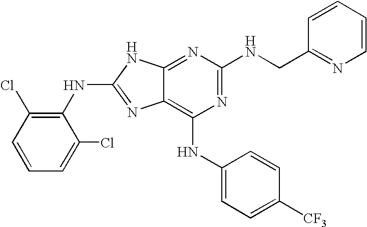

### Example 26

### {3,5-Dichloro-4-[6-(4-trifluoromethyl-Phenylamino)-9H-purin-8-ylamino]-phenyl}-methanol

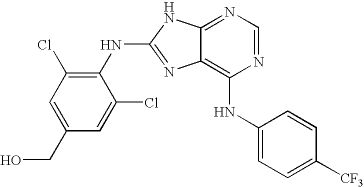

### Biological Testing:

Functional assay: block of capsaicin-induced Ca²⁺ influx

### A. Human Assay

HEK293 cells were transfected with human TRPV1 cloned in pcDNA3.1zeo(+) using the Effectene non-liposomal lipid based transfection kit (Qiagen) (hTRPV1/HEK293). hTRPV1/HEK293 cells were routinely grown as monolayers under selection in zeocin (200 μg/mL; Invitrogen) in Dulbecco's Modified Eagle Medium (DMEM, Gibco BRL) supplemented with 10% fetal bovine serum, and penicillin/streptomycin (50 units/mL) in 5% CO₂ at 37° C. Cells were passaged frequently, every 3-5 days, to avoid overgrowth, depletion of essential medium components, or acidic medium exposure. Cells were passaged using a brief wash in 0.05% trypsin with 1 mM EDTA, followed by dissociation in divalent-free phosphate-buffered saline (Hyclone #SH30028.02). Dissociated cells were seeded onto poly-D-lysine coated black-walled 96-well plates (Biocoat; Becton Dickinson #354640) at about 40,000 cells per well and grown for approximately 1 day in culture medium to near confluency. The assay buffer was composed of 130 mM NaCl, 2 mM KCl, 2 mM MgCl₂, 10 mM HEPES, 5 mM glucose, and either 2 mM or 20 μM CaCl₂. On the day of the experiment, the culture medium was replaced with 2 mM calcium assay buffer using an automated plate washer (EL×405; Biotek, VT). The cells were incubated in 100 μL/well Fluo-3/AM (2 μM; TEFLabs #0116) with Pluronic F127 (100 μg/mL; Sigma #P2443) for 1 h at rt in the dark. After loading the cells, the dye solution was replaced with 50 μL/well of 20 μM calcium assay buffer using the EL×405 plate washer. Test compounds (50 μL/well) were added to the plate and incubated for 30 min. Intracellular Ca²⁺ levels were subsequently assayed using a Fluorometric Imaging Plate Reader (FLIPR™ instrument, Molecular Devices, CA) to simultaneously monitor Fluo-3 fluorescence in all wells (λ_{excitation}=488 nm, λₑₘᵢₛₛᵢₒₙ=540 nm) during challenge with agonist (capsaicin). The IC₅₀ values were determined. Cells were challenged with 150 nM capsaicin and the fluorescence counts were captured following agonist addition at a sampling rate of 0.33 Hz. The contents of the wells were mixed 3 times (40 μL mix volume) immediately after the additions were made. Concentration-dependence of block was determined by exposing each well of cells in duplicate rows of a 96-well plate to a serial dilution of test compound. The concentration series usually started at 10 μM with a three-fold serial decrement in concentration. The magnitude of the capsaicin response was determined by measuring the change in fluo3 fluorescence before and 100 seconds after the addition of the agonist. Data were analyzed using a non-linear regression program (Origin; OriginLab, MA).

### B. Rat Assay

This assay was performed similarly to the human assay described above, but using HEK293 cells transfected with rat TRPV1 (rTRPV1/HEK293). These cells had a geneticin selection marker and were grown in Dulbecco's Modified Eagle Medium (DMEM, Gibco BRL) supplemented with 10% fetal bovine serum, penicillin/streptomycin (50 units/mL), and 500 μg/mL geneticin in 5% CO₂ at 37° C.

Results for the compounds tested in these assays are presented in Table 1. IC₅₀ values shown are the average (mean) of the results obtained. Where activity is shown as greater than (>) a particular value, the value is the solubility limit of the compound in the assay medium.

TABLE 1

| | Human | Rat |
|---|---|---|
| Ex. | IC₅₀ (nM) | IC₅₀ (nM) |
| 1 | 17 | 4.9 |
| 2 | NA | 19 |
| 3 | 2.3 | 0.15 |
| 4 | 3390 | 1750 |
| 5 | 170 | 18 |
| 6 | 29 | 3 |
| 7 | >6670 | >6670 |
| 8 | 3070 | 1010 |
| 9 | 2300 | 1050 |
| 10 | 1460 | 1130 |

NA = data not available

While the invention has been illustrated by reference to exemplary and preferred embodiments, it will be understood that the invention is intended not to be limited by the foregoing detailed description, but to be defined by the appended claims as properly construed under principles of patent law.

What is claimed is:

1. A composition of matter selected from the group consisting of:
(a) compounds of Formula (I): 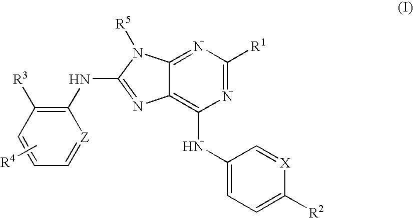
wherein:
R¹ is —H, —C₁₋₆alkyl, —OC₁₋₆alkyl, —NR^{a}R^{b}, —S—C₁₋₆alkyl, or —SO₂—C₁₋₆alkyl;
where R^{a} and R^{b} are each independently —H, —C₁₋₆alkyl, or —CH₂-pyridinyl; or, R^{a} and R^{b} taken together with the nitrogen of attachment in —NR^{a}R^{b} form a saturated monocyclic heterocycloalkyl group unsubstituted or substituted with a —C₁₋₆alkyl substituent;
R² is —H, —C₁₋₆alkyl, —OH, —OC₁₋₆alkyl, —CN, —NO₂, —N(R^{h})Rⁱ, —C(O)N(R^{h})Rⁱ, —N(R^{h})C(O)Rⁱ, —N(R^{h})SO₂C₁₋₆alkyl, —N(SO₂C₁₋₆alkyl)₂, —C(O)C₁₋₆alkyl, —S(O)₀₋₂—C₁₋₆alkyl, —SO₂CF₃, —SO₂N(R^{h})Rⁱ, —SCF₃, halo, —CF₃, —OCF₃, —CO₂H, —CO₂C₁₋₆alkyl, —C(R^{j})₂—CN, —C(R^{j})₂—CO₂C₁₋₄alkyl, —C(R^{j})₂—CO₂H, —C(R^{j})₂—CON(R^{h})Rⁱ, —C(R^{j})₂—CH₂N(R^{h})Rⁱ, or —C(R^{j})₂—OH;
where R^{h} and Rⁱ are each independently —H or —C₁₋₆alkyl; or R^{h} and Rⁱ taken together with their nitrogen of attachment in —NR^{h}Rⁱ form a saturated monocyclic heterocycloalkyl group unsubstituted or substituted with methyl;
where each R^{j} is independently —H or —C₁₋₆alkyl;
X and Z are each independently N or CR^{m}, where R^{m} is —H, halo, or —CF₃;
R³ is —CF₃, halo, —CN, —CO₂H, —CO₂C₁₋₆alkyl, —C(O)N(R^{k})R^{l}, —C₁₋₄alkyl-OH, —C₁₋₄alkyl-N(R^{k})R^{l}, —S(O)₀₋₂—C₁₋₆alkyl, —SO₂CF₃, or —SO₂N(R^{k})R^{l};
where R^{k} and R^{l} are each independently —H or —C₁₋₆alkyl;
R⁴ is —H, —CF₃, halo, —CN, —CO₂H, —CO₂C₁₋₆alkyl, —C(O)N(Rⁿ)R^{o}, —C₁₋₄alkyl-OH, —C₁₋₄alkyl-N(Rⁿ)R^{o}, —S(O)₀₋₂—C₁₋₆alkyl, —SO₂CF₃, or —SO₂N(Rⁿ)R^{o};
where Rⁿ and R^{o} are each independently —H or —C₁₋₆alkyl; and
R⁵ is —H or —CH₃;
and (b) pharmaceutically acceptable salts of the compounds of Formula (I), pharmaceutically acceptable prodrugs of the compounds of Formula (I), and pharmaceutically active metabolites of the compounds of Formula (I).

2. A composition of matter as defined in claim 1 selected from the group consisting of:
(a) the compounds of Formula (I) wherein R¹ is —H, methyl, methanesulfanyl, methanesulfonyl, or methoxy;
and (b) pharmaceutically acceptable salts of said compounds.

3. A composition of matter as defined in claim 1 selected from the group consisting of:
(a) the compounds of Formula (I) wherein R¹ is isopropylamino, isobutylamino, or (pyridin-2-ylmethyl)amino, or a pyrrolidin-1-yl, piperidin-1-yl, morpholin-4-yl, or piperazin-1-yl group unsubstituted or substituted with a —C₁₋₄alkyl substituent;
and (b) pharmaceutically acceptable salts of said compounds.

4. A composition of matter as defined in claim 1 selected from the group consisting of:
(a) the compounds of Formula (I) wherein R² is —H, methyl, isopropyl, tert-butyl, —OCH₃, —SO₂CH₃, —SO₂CF₃, —SO₂NH₂, —SO₂(morpholinyl), —SO₂(piperazinyl), fluoro, chloro, —CF₃, —OCF₃, —CO₂CH₃, —C(CH₃)₂—CN, —C(CH₃)₂—CO₂CH₃, —C(PH₃)₂—CONH₂, or —C(CH₃)₂—OH;
and (b) pharmaceutically acceptable salts of said compounds.

5. A composition of matter as defined in claim 1 selected from the group consisting of:
(a) the compounds of Formula (I) wherein R² is —H, —CF₃, tert-butyl, or methanesulfonyl;
and (b) pharmaceutically acceptable salts of said compounds.

6. A composition of matter as defined in claim 1 selected from the group consisting of:
(a) the compounds of Formula (I) wherein R² is —CF₃;
and (b) pharmaceutically acceptable salts of said compounds.

7. A composition of matter as defined in claim 1 selected from the group consisting of:
(a) the compounds of Formula (I) wherein X is CR^{m}, where R^{m} is —H, chloro, or fluoro;
and (b) pharmaceutically acceptable salts of said compounds.

8. A composition of matter as defined in claim 1 selected from the group consisting of:
(a) the compounds of Formula (I) wherein X is CR^{m}, where R^{m} is —H;
and (b) pharmaceutically acceptable salts of said compounds.

9. A composition of matter as defined in claim 1 selected from the group consisting of:
(a) the compounds of Formula (I) wherein Z is CR^{m}, where R^{m} is —H, chloro, or —CF₃.
and (b) pharmaceutically acceptable salts of said compounds.

10. A composition of matter as defined in claim 1 selected from the group consisting of:
(a) the compounds of Formula (I) wherein R³ is —CF₃, halo, —CN, —C(O)N(R^{k})R^{l}, —CH₂OH, or —CH₂N(R^{k})R^{l};
and (b) pharmaceutically acceptable salts of said compounds.

11. A composition of matter as defined in claim 1 selected from the group consisting of:
(a) the compounds of Formula (I) wherein R³ is —CF₃ or halo;
and (b) pharmaceutically acceptable salts of said compounds.

12. A composition of matter as defined in claim 1 selected from the group consisting of:
(a) the compounds of Formula (I) wherein R⁴ is —H, —CN, —C(O)N(R^{k})R^{l}, —CH₂OH, or —CH₂N(R^{k})R^{l};
and (b) pharmaceutically acceptable salts of said compounds.

13. A composition of matter as defined in claim 1 selected from the group consisting of:
(a) the compounds of Formula (I) wherein R⁴ is —H;
and (b) pharmaceutically acceptable salts of said compounds.

14. A composition of matter as defined in claim 1 selected from the group consisting of:
(a) the compounds of Formula (I) wherein R⁵ is —H;
and (b) pharmaceutically acceptable salts of said compounds.

15. A composition of matter as defined in claim 1 selected from the group consisting of:
(a) the compounds of Formula (I) wherein R^{a} and R^{b} are each independently —H, methyl, ethyl, isopropyl, isobutyl, or pyridinylmethyl;
and (b) pharmaceutically acceptable salts of said compounds.

16. A composition of matter as defined in claim 1 selected from the group consisting of:
(a) the compounds of Formula (I) wherein R^{a} and R^{b} taken together with the nitrogen of attachment form an azetidinyl, pyrrolidinyl, piperidinyl, 2-oxo-piperidin-1-yl, piperazinyl, oxo-piperazinyl, morpholinyl, thiomorpholinyl, 1,1-dioxo-1λ⁶-thiomorpholin-4-yl, or azepanyl group unsubstituted or substituted with a —C₁₋₄alkyl substituent;
and (b) pharmaceutically acceptable salts of said compounds.

17. A composition of matter as defined in claim 1 selected from the group consisting of:
(a) the compounds of Formula (I) wherein R^{a} and R^{b} taken together with the nitrogen of attachment form an azetidinyl, pyrrolidinyl, piperidinyl, piperazinyl, or morpholinyl group, each unsubstituted or substituted with a methyl, isopropyl, or isobutyl substituent;
and (b) pharmaceutically acceptable salts of said compounds.

18. A composition of matter as defined in claim 1, selected from the group consisting of:
N⁸-(2,6-Dichloro-phenyl)-N⁶-(4-trifluoromethyl-phenyl)-9H-purine-6,8-diamine;
N⁸-(2,6-Dichloro-phenyl)-N⁶-(6-trifluoromethyl-pyridin-3-yl)-9H-purine-6,8-diamine;
N⁶-(4-tert-Butyl-phenyl)-N⁸-(2,6-dichloro-phenyl)-9H-purine-6,8-diamine;
N⁸-(2,6-Dichloro-phenyl)-N⁶-phenyl-9H-purine-6,8-diamine;
N⁸-(2,6-Dichloro-phenyl)-N⁶-(4-methanesulfonyl-phenyl)-9H-purine-6,8-diamine;
N⁸-(2,6-Dichloro-phenyl)-2-methyl-N⁶-(4-trifluoromethyl-phenyl)-9H-purine-6,8-diamine;
N⁸-(2-Chloro-phenyl)-N⁶-(4-trifluoromethyl-phenyl)-9H-purine-6,8-diamine;
N⁶-(4-Trifluoromethyl-phenyl)-N⁸-(2-trifluoromethyl-phenyl)-9H-purine-6,8-diamine;
N⁸-(2-Trifluoromethyl-phenyl)-N⁶-(6-trifluoromethyl-pyridin-3-yl)-9H-purine-6,8-diamine;
N⁸-(2,6-Dichloro-phenyl)-9-methyl-N⁶-(4-trifluoromethyl-phenyl)-9H-purine-6,8-diamine;
N⁶-(4-Trifluoromethyl-phenyl)-N⁸-(3-trifluoromethyl-pyridin-2-yl)-9H-purine-6,8-diamine;
N⁸-(2,6-Dichloro-phenyl)-2-methylsulfanyl-N⁶-(4-trifluoromethyl-phenyl)-9H-purine-6,8-diamine;
N⁸-(2,6-Dichloro-phenyl)-2-methoxy-N⁶-(4-trifluoromethyl-phenyl)-9H-purine-6,8-diamine;
N⁸-(2,6-Dichloro-phenyl)-2-methanesulfonyl-N⁶-(4-trifluoromethyl-phenyl)-9H-purine-6,8-diamine;
N⁸-(2,6-Dichloro-phenyl)-2-morpholin-4-yl-N⁶-(4-trifluoromethyl-phenyl)-9H-purine-6,8-diamine;
N⁸-(2,6-Dichloro-phenyl)-2-(4-methyl-piperazin-1-yl)-N⁶-(4-trifluoromethyl-phenyl)-9H-purine-6,8-diamine;
N⁸-(2,6-Dichloro-phenyl)-2-(4-isobutyl-piperazin-1-yl)-N⁶-(4-trifluoromethyl-phenyl)-9H-purine-6,8-diamine;
N⁸-(2,6-Dichloro-phenyl)-2-piperidin-1-yl-N⁶-(4-trifluoromethyl-phenyl)-9H-purine-6,8-diamine;
N⁸-(2,6-Dichloro-phenyl)-N²-isobutyl-N⁶-(4-trifluoromethyl-phenyl)-9H-purine-2,6,8-triamine;
N⁸-(2,6-Dichloro-phenyl)-N²-isopropyl-N⁶-(4-trifluoromethyl-phenyl)-9H-purine-2,6,8-triamine;
N⁸-(2,6-Dichloro-phenyl)-2-pyrrolidin-1-yl-N⁶-(4-trifluoromethyl-phenyl)-9H-purine-6,8-diamine;
N⁸-(2,6-Dichloro-phenyl)-N⁶-(3-fluoro-4-trifluoromethyl-phenyl)-9H-purine-6,8-diamine;
N⁶-(3-Chloro-4-trifluoromethyl-phenyl)-N⁸-(2,6-dichloro-phenyl)-9H-purine-6,8-diamine;
N⁸-(2,6-Dichloro-phenyl)-N⁶-(3-fluoro-4-methanesulfonyl-phenyl)-9H-purine-6,8-diamine;
N⁸-(2,6-Dichloro-phenyl)-N²-pyridin-2-ylmethyl-N⁶-(4-trifluoromethyl-phenyl)-9H-purine-2,6,8-triamine; and
{3,5-Dichloro-4-[6-(4-trifluoromethyl-phenylamino)-9H-purin-8-ylamino]-phenyl}-methanol;
and pharmaceutically acceptable salts thereof.

19. A pharmaceutical composition for treating a disease, disorder, or medical condition mediated by TRPV1 activity, comprising:
(a) an effective amount of at least one agent selected from compounds of Formula (I) and pharmaceutically acceptable salts, pharmaceutically acceptable prodrugs, and pharmaceutically active metabolites of said compounds of Formula (I): 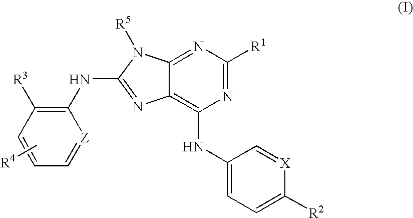
wherein:
R¹ is —H, —C₁₋₆alkyl, —OC₁₋₆alkyl, —NR^{a}R^{b}, —S—C₁₋₆alkyl, or —SO₂—C₁₋₆alkyl;
where R^{a} and R^{b} are each independently —H, —C₁₋₆alkyl, or —CH₂-pyridinyl; or, R^{a} and R^{b} taken together with the nitrogen of attachment in —NR^{a}R^{b} form a saturated monocyclic heterocycloalkyl group unsubstituted or substituted with a —C₁₋₆alkyl substituent;
R² is —H, —C₁₋₆alkyl, —OH, —OC₁₋₆alkyl, —CN, —NO₂, —N(R^{h})Rⁱ, —C(O)N(R^{h})Rⁱ, —N(R^{h})C(O)Rⁱ, —N(R^{h})SO₂C₁₋₆alkyl, —N(SO₂C₁₋₆alkyl)₂, —C(O)C₁₋₆alkyl, —S(O)₀₋₂—C₁₋₆alkyl, —SO₂CF₃, —SO₂N(R^{h})Rⁱ, —SCF₃, halo, —CF₃, —OCF₃, —CO₂H, —CO₂C₁₋₆alkyl, —C(R^{j})₂—CN, —C(R^{j})₂—CO₂C₁₋₄alkyl, —C(R^{j})₂—CO₂H, —C(R^{j})₂—CON(R^{h})Rⁱ, —C(R^{j})₂—CH₂N(R^{h})Rⁱ, or —C(R^{j})₂—OH;
where R^{h} and Rⁱ are each independently —H or —C₁₋₆alkyl; or R^{h} and Rⁱ taken together with their nitrogen of attachment in —NR^{h}Rⁱ form a saturated monocyclic heterocycloalkyl group unsubstituted or substituted with methyl;
where each R^{j} is independently-H or —C₁₋₆alkyl;
X and Z are each independently N or CR^{m}, where R^{m} is —H, halo, or —CF₃;
R³ is —CF₃, halo, —CN, —CO₂H, —CO₂C₁₋₆alkyl, —C(O)N(R^{k})R^{l}, —C₁₋₄alkyl-OH, —C₁₋₄alkyl-N(R^{k})R^{l}, —S(O)₀₋₂—C₁₋₆alkyl, —SO₂CF₃, or —SO₂N(R^{k})R^{l};
where R^{k} and R^{l} are each independently —H or —C₁₋₆alkyl;
R⁴ is —H, —CF₃, halo, —CN, —CO₂H, —CO₂C₁₋₆alkyl, —C(O)N(Rⁿ)R^{o}, —C₁₋₄alkyl-OH, —C₁₋₄alkyl-N(Rⁿ)R^{o}, —S(O)₀₋₂—C₁₋₆alkyl, —SO₂CF₃, or —SO₂N(Rⁿ)R^{o};
where Rⁿ and R^{o} are each independently —H or —C₁₋₆alkyl; and
R⁵ is —H or —CH₃;
and (b) a pharmaceutically acceptable excipient.

20. A pharmaceutical composition according to claim 19, wherein said agent is selected from the group consisting of:
N⁸-(2,6-Dichloro-phenyl)-N⁶-(4-trifluoromethyl-phenyl)-9H-purine-6,8-diamine;
N⁸-(2,6-Dichloro-phenyl)-N⁶-(6-trifluoromethyl-pyridin-3-yl)-9H-purine-6,8-diamine;
N⁶-(4-tert-Butyl-phenyl)-N⁸-(2,6-dichloro-phenyl)-9H-purine-6,8-diamine;
N⁸-(2,6-Dichloro-phenyl)-N⁶-phenyl-9H-purine-6,8-diamine;
N⁸-(2,6-Dichloro-phenyl)-N⁶-(4-methanesulfonyl-phenyl)-9H-purine-6,8-diamine;
N⁸-(2,6-Dichloro-phenyl)-2-methyl-N⁶-(4-trifluoromethyl-phenyl)-9H-purine-6,8-diamine;
N⁸-(2-Chloro-phenyl)-N⁶-(4-trifluoromethyl-phenyl)-9H-purine-6,8-diamine;
N⁶-(4-Trifluoromethyl-phenyl)-N⁸-(2-trifluoromethyl-phenyl)-9H-purine-6,8-diamine;
N⁸-(2-Trifluoromethyl-phenyl)-N⁶-(6-trifluoromethyl-pyridin-3-yl)-9H-purine-6,8-diamine;
N⁸-(2,6-Dichloro-phenyl)-9-methyl-N⁶-(4-trifluoromethyl-phenyl)-9H-purine-6,8-diamine;
N⁶-(4-Trifluoromethyl-phenyl)-N⁸-(3-trifluoromethyl-pyridin-2-yl)-9H-purine-6,8-diamine;
N⁸-(2,6-Dichloro-phenyl)-2-methylsulfanyl-N⁶-(4-trifluoromethyl-phenyl)-9H-purine-6,8-diamine;
N⁸-(2,6-Dichloro-phenyl)-2-methoxy-N⁶-(4-trifluoromethyl-phenyl)-9H-purine-6,8-diamine;
N⁸-(2,6-Dichloro-phenyl)-2-methanesulfonyl-N⁶-(4-trifluoromethyl-phenyl)-9H-purine-6,8-diamine;
N⁸-(2,6-Dichloro-phenyl)-2-morpholin-4-yl-N⁶-(4-trifluoromethyl-phenyl)-9H-purine-6,8-diamine;
N⁸-(2,6-Dichloro-phenyl)-2-(4-methyl-piperazin-1-yl)-N⁶-(4-trifluoromethyl-phenyl)-9H-purine-6,8-diamine;
N⁸-(2,6-Dichloro-phenyl)-2-(4-isobutyl-piperazin-1-yl)-N⁶-(4-trifluoromethyl-phenyl)-9H-purine-6,8-diamine;
N⁸-(2,6-Dichloro-phenyl)-2-piperidin-1-yl-N⁶-(4-trifluoromethyl-phenyl)-9H-purine-6,8-diamine;
N⁸-(2,6-Dichloro-phenyl)-N²-isobutyl-N⁶-(4-trifluoromethyl-phenyl)-9H-purine-2,6,8-triamine;
N⁸-(2,6-Dichloro-phenyl)-N²-isopropyl-N⁶-(4-trifluoromethyl-phenyl)-9H-purine-2,6,8-triamine;
N⁸-(2,6-Dichloro-phenyl)-2-pyrrolidin-1-yl-N⁶-(4-trifluoromethyl-phenyl)-9H-purine-6,8-diamine;
N⁸-(2,6-Dichloro-phenyl)-N⁶-(3-fluoro-4-trifluoromethyl-phenyl)-9H-purine-6,8-diamine;
N⁶-(3-Chloro-4-trifluoromethyl-phenyl)-N⁸-(2,6-dichloro-phenyl)-9H-purine-6,8-diamine;
N⁸-(2,6-Dichloro-phenyl)-N⁶-(3-fluoro-4-methanesulfonyl-phenyl)-9H-purine-6,8-diamine;
N⁸-(2,6-Dichloro-phenyl)-N²-pyridin-2-ylmethyl-N⁶-(4-trifluoromethyl-phenyl)-9H-purine-2,6,8-triamine; and
{3,5-Dichloro-4-[6-(4-trifluoromethyl-phenylamino)-9H-purin-8-ylamino]-phenyl}-methanol;
and pharmaceutically acceptable salts thereof.

21. A method of treating a subject suffering from or diagnosed with a disease, disorder, or condition mediated by TRPV1 activity, comprising administering to the subject an effective amount of at least one agent selected from compounds of Formula (I) and pharmaceutically acceptable salts, pharmaceutically acceptable prodrugs, and pharmaceutically active metabolites of said compounds of Formula (I): 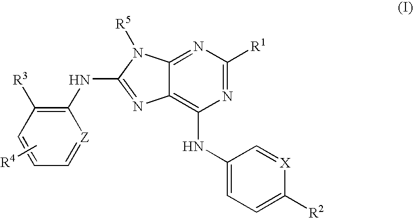
wherein:
R¹ is —H, —C₁₋₆alkyl, —OC₁₋₆alkyl, —NR^{a}R^{b}, —S—C₁₋₆alkyl, or —SO₂—C₁₋₆alkyl;
where R^{a} and R^{b} are each independently —H, —C₁₋₆alkyl, or —CH₂-pyridinyl; or, R^{a} and R^{b} taken together with the nitrogen of attachment in —NR^{a}R^{b} form a saturated monocyclic heterocycloalkyl group unsubstituted or substituted with a —C₁₋₆alkyl substituent;
R² is —H, —C₁₋₆alkyl, —OH, —OC₁₋₆alkyl, —CN, —NO₂, —N(R^{h})Rⁱ, —C(O)N(R^{h})Rⁱ, —N(R^{h})C(O)Rⁱ, —N(R^{h})SO₂C₁₋₆alkyl, —N(SO₂C₁₋₆alkyl)₂, —C(O)C₁₋₆alkyl, —S(O)₀₋₂—C₁₋₆alkyl, —SO₂CF₃, —SO₂N(R^{h})Rⁱ, —SCF₃, halo, —CF₃, —OCF₃, —CO₂H, —CO₂C₁₋₆alkyl, —C(R^{j})₂—CN, —C(R)₂—CO₂C₁₋₄alkyl, —C(R^{j})₂—CO₂H, —C(R^{j})₂—CON(R^{h})Rⁱ, —C(R^{j})₂—CH₂N(R^{h})Rⁱ, or —C(R^{j})₂—OH;
where R^{h} and Rⁱ are each independently —H or —C₁₋₆alkyl; or R^{h} and Rⁱ taken together with their nitrogen of attachment in —NR^{h}Rⁱ form a saturated monocyclic heterocycloalkyl group unsubstituted or substituted with methyl;
where each R^{j} is independently —H or —C₁₋₆alkyl;
X and Z are each independently N or CR^{m}, where R^{m} is —H, halo, or —CF₃;
R³ is —CF₃, halo, —CN, —CO₂H, —CO₂C₁₋₆alkyl, —C(O)N(R^{k})R^{l}, —C₁₋₄alkyl-OH, —C₁₋₄alkyl-N(R^{k})R^{l}, —S(O)₀₋₂—C₁₋₆alkyl, —SO₂CF₃, or —SO₂N(R^{k})R^{l};
where R^{k} and R^{l} are each independently —H or —C₁₋₆alkyl;
R⁴ is —H, —CF₃, halo, —CN, —CO₂H, —CO₂C₁₋₆alkyl, —C(O)N(Rⁿ)R^{o}, —C₁₋₄alkyl-OH, —C₁₋₄alkyl-N(Rⁿ)R^{o}, —S(O)₀₋₂—C₁₋₆alkyl, —SO₂CF₃, or —SO₂N(Rⁿ)R^{o};
where Rⁿ and R^{o} are each independently —H or —C₁₋₆alkyl; and
R⁵ is —H or —CH₃.

22. A method according to claim 21, wherein said agent is selected from the group consisting of:
N⁸-(2,6-Dichloro-phenyl)-N⁶-(4-trifluoromethyl-phenyl)-9H-purine-6,8-diamine;
N⁸-(2,6-Dichloro-phenyl)-N⁶-(6-trifluoromethyl-pyridin-3-yl)-9H-purine-6,8-diamine;
N⁶-(4-tert-Butyl-phenyl)-N⁸-(2,6-dichloro-phenyl)-9H-purine-6,8-diamine;
N⁸-(2,6-Dichloro-phenyl)-N⁶-phenyl-9H-purine-6,8-diamine;
N⁸-(2,6-Dichloro-phenyl)-N⁶-(4-methanesulfonyl-phenyl)-9H-purine-6,8-diamine;
N⁸-(2,6-Dichloro-phenyl)-2-methyl-N⁶-(4-trifluoromethyl-phenyl)-9H-purine-6,8-diamine;
N⁸-(2-Chloro-phenyl)-N⁶-(4-trifluoromethyl-phenyl)-9H-purine-6,8-diamine;
N⁶-(4-Trifluoromethyl-phenyl)-N⁸-(2-trifluoromethyl-phenyl)-9H-purine-6,8-diamine;
N⁸-(2-Trifluoromethyl-phenyl)-N⁶-(6-trifluoromethyl-pyridin-3-yl)-9H-purine-6,8-diamine;
N⁸-(2,6-Dichloro-phenyl)-9-methyl-N⁶-(4-trifluoromethyl-phenyl)-9H-purine-6,8-diamine;
N⁶-(4-Trifluoromethyl-phenyl)-N⁸-(3-trifluoromethyl-pyridin-2-yl)-9H-purine-6,8-diamine;
N⁸-(2,6-Dichloro-phenyl)-2-methylsulfanyl-N⁶-(4-trifluoromethyl-phenyl)-9H-purine-6,8-diamine;
N⁸-(2,6-Dichloro-phenyl)-2-methoxy-N⁶-(4-trifluoromethyl-phenyl)-9H-purine-6,8-diamine;
N⁸-(2,6-Dichloro-phenyl)-2-methanesulfonyl-N⁶-(4-trifluoromethyl-phenyl)-9H-purine-6,8-diamine;
N⁸-(2,6-Dichloro-phenyl)-2-morpholin-4-yl-N⁶-(4-trifluoromethyl-phenyl)-9H-purine-6,8-diamine;
N⁸-(2,6-Dichloro-phenyl)-2-(4-methyl-piperazin-1-yl)-N⁶-(4-trifluoromethyl-phenyl)-9H-purine-6,8-diamine;
N⁸-(2,6-Dichloro-phenyl)-2-(4-isobutyl-piperazin-1-yl)-N⁶-(4-trifluoromethyl-phenyl)-9H-purine-6,8-diamine;
N⁸-(2,6-Dichloro-phenyl)-2-piperidin-1-yl-N⁶-(4-trifluoromethyl-phenyl)-9H-purine-6,8-diamine;
N⁸-(2,6-Dichloro-phenyl)-N²-isobutyl-N⁶-(4-trifluoromethyl-phenyl)-9H-purine-2,6,8-triamine;
N⁸-(2,6-Dichloro-phenyl)-N²-isopropyl-N⁶-(4-trifluoromethyl-phenyl)-9H-purine-2,6,8-triamine;
N⁸-(2,6-Dichloro-phenyl)-2-pyrrolidin-1-yl-NB-(4-trifluoromethyl-phenyl)-9H-purine-6,8-diamine;
N⁸-(2,6-Dichloro-phenyl)-N⁶-(3-fluoro-4-trifluoromethyl-phenyl)-9H-purine-6,8-diamine;
N⁶-(3-Chloro-4-trifluoromethyl-phenyl)-N⁸-(2,6-dichloro-phenyl)-9H-purine-6,8-diamine;
N⁸-(2,6-Dichloro-phenyl)-N⁶-(3-fluoro-4-methanesulfonyl-phenyl)-9H-purine-6,8-diamine;
N⁸-(2,6-Dichloro-phenyl)-N²-pyridin-2-ylmethyl-N⁶-(4-trifluoromethyl-phenyl)-9H-purine-2,6,8-triamine; and
{3,5-Dichloro-4-[6-(4-trifluoromethyl-phenylamino)-9H-purin-8-ylamino]-phenyl}-methanol;
and pharmaceutically acceptable salts thereof.

23. A method according to claim 21, wherein the disease, disorder, or condition is pain; itch or an inflammatory disorder; an inner ear disorder; fever or another condition or disorder of thermoregulation; tracheobronchial or diaphragmatic dysfunction; a gastrointestinal or urinary tract disorder; or a disorder associated with reduced blood flow to the central nervous system or CNS hypoxia.

24. A method according to claim 21, wherein the disease, disorder, or condition is pain, arthritis, itch, cough, asthma, inflammatory bowel disease, or an inner ear disorder.

25. A process for the preparation of a compound of Formula (I) or a pharmaceutically acceptable salt thereof: 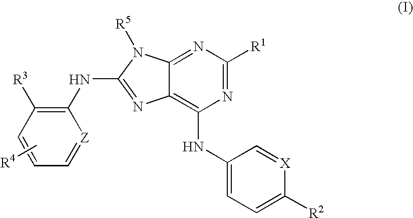
comprising reacting a compound of formula (VIII): 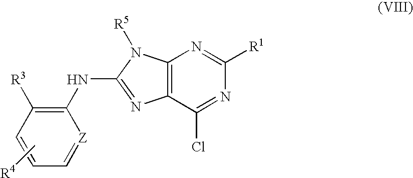
with an aromatic amine of formula (IX): 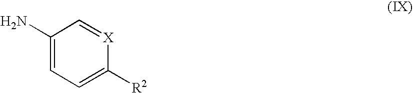
to give a compound of Formula (I);
wherein:
R^{1 is —H, —C}₁₋₆alkyl, —OC₁₋₆alkyl, —NR^{a}R^{b}, —S—C₁₋₆alkyl, or —SO₂—C₁₋₆alkyl;
where R^{a} and R^{b} are each independently —H, —C₁₋₆alkyl, or —CH₂-pyridinyl; or, R^{a} and R^{b} taken together with the nitrogen of attachment in —NR^{a}R^{b} form a saturated monocyclic heterocycloalkyl group unsubstituted or substituted with a —C₁₋₆alkyl substituent;
R² is —H, —C₁₋₆alkyl, —OH, —OC₁₋₆alkyl, —CN, —NO₂, —N(R^{h})Rⁱ, —C(O)N(R^{h})Rⁱ, —N(R^{h})C(O)Rⁱ, —N(R^{h})SO₂C₁₋₆alkyl, —N(SO₂C₁₋₆alkyl)₂, —C(O)C₁₋₆alkyl, —S(O)₀₋₂—C₁₋₆alkyl, —SO₂CF₃, —SO₂N(R^{h})Rⁱ, —SCF₃, halo, —CF₃, —OCF₃, —CO₂H, —CO₂C₁₋₆alkyl, —C(R^{j})₂—CN, —C(R^{j})₂—CO₂C₁₋₄alkyl, —C(R^{j})₂—CO₂H, —C(R^{j})₂—CON(R^{h})R^{j}, —C(R^{j})₂—CH₂N(R^{h})Rⁱ, or —C(R^{j})₂—OH;
where R^{h} and Rⁱ are each independently —H or —C₁₋₆alkyl; or R^{h} and Rⁱ taken together with their nitrogen of attachment in —NR^{h}Rⁱ form a saturated monocyclic heterocycloalkyl group unsubstituted or substituted with methyl;
where each R^{j} is independently —H or —C₁₋₆alkyl;
X and Z are each independently N or CR^{m}, where R^{m} is —H, halo, or —CF₃;
R³ is —CF₃, halo, —CN, —CO₂H, —CO₂C₁₋₆alkyl, —C(O)N(R^{k})R^{l}, —C₁₋₄alkyl-OH, —C₁₋₄alkyl-N(R^{k})R^{l}, —S(O)₀₋₂—C₁₋₆alkyl, —SO₂CF₃, or —SO₂N(R^{k})R^{l};
where R^{k} and R^{l} are each independently —H or —C₁₋₆alkyl;
R⁴ is —H, —CF₃, halo, —CN, —CO₂H, —CO₂C₁₋₆alkyl, —C(O)N(Rⁿ)R^{o}, —C₁₋₄alkyl-OH, —C₁₋₄alkyl-N(Rⁿ)R^{o}, —S(O)₀₋₂—C₁₋₆alkyl, —SO₂CF₃, or —SO₂N(Rⁿ)R^{o};
where Rⁿ and R^{o} are each independently —H or —C₁₋₆alkyl; and
R⁵ is —H or —CH₃.

26. A process according to claim 25, further comprising reacting a compound of formula (VI): 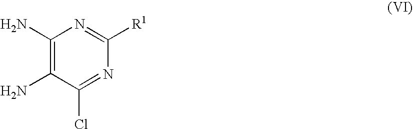
with an isothiocyanate of formula (VII): 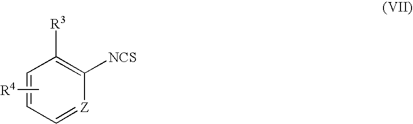
to give a compound of formula (VIII).

27. A process according to claim 26, further comprising reacting a dichloro-pyrimidine of formula (V): 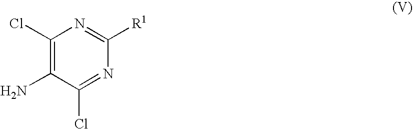
with ammonia or an ammonia equivalent to give a diaminopyrimidine of formula (VI).

28. A compound of formula (VIII): 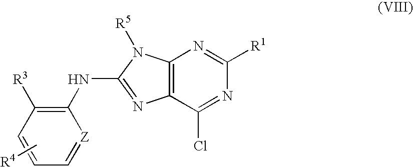
wherein:
R¹ is —H, —C₁₋₆alkyl, —OC₁₋₆alkyl, —NR^{a}R^{b}, —S—C₁₋₆alkyl, or —SO₂—CR₁₋₆alkyl;
where R^{a} and R^{b} are each independently —H, —C₁₋₆alkyl, or —CH₂-pyridinyl; or, R^{a} and R^{b} taken together with the nitrogen of attachment in —NR^{a}R^{b} form a saturated monocyclic heterocycloalkyl group unsubstituted or substituted with a —C₁₋₆alkyl substituent;
R³ is —CF₃, halo, —CN, —CO₂H, —CO₂C₁₋₆alkyl, —C(O)N(R^{k})R^{l}, —C₁₋₄alkyl-OH, —C₁₋₄alkyl-N(R^{k})R^{l}, —S(O)₀₋₂—C₁₋₆alkyl, —SO₂CF₃, or —SO₂N(R^{k})R^{l};
where R^{k} and R^{l} are each independently —H or —C₁₋₆alkyl;
R⁴ is —H, —CF₃, halo, —CN, —CO₂H, —CO₂C₁₋₆alkyl, —C(O)N(Rⁿ)R^{o}, —C₁₋₄alkyl-OH, —C₁₋₄alkyl-N(Rⁿ)R^{o}, —S(O)₀₋₂—C₁₋₆alkyl, —SO₂CF₃, or —SO₂N(Rⁿ)R^{o};
where Rⁿ and R^{o} are each independently —H or —C₁₋₆alkyl; and
R⁵ is —H or —CH₃.
